SEQUENCE LISTING (31 sequences)

SEQ ID NO: 1 - DNA, 19 bp, Artificial Sequence
  DENV2-F3 primer
cacactggatagcagcttc

SEQ ID NO: 2 - DNA, 20 bp, Artificial Sequence
  DENV2-B3 primer
ctatgtccaggatgttgctc

SEQ ID NO: 3 - DNA, 38 bp, Artificial Sequence
  DENV2-FIP primer
ggccaccactgtgaggatgagaacaccccaagataacc

SEQ ID NO: 4 - DNA, 41 bp, Artificial Sequence
  DENV2-BIP primer
caaacgagatgggtttcctggattgctgggttgtaatgctt

SEQ ID NO: 5 - DNA, 21 bp, Artificial Sequence
  DENV2-LoopF primer
ggctatgacaacgtaggtcaa

SEQ ID NO: 6 - DNA, 22 bp, Artificial Sequence
  DENV2-LoopB primer
acgaagaaagatctcggattgg

SEQ ID NO: 7 - DNA, 20 bp, Artificial Sequence
  SARS-CoV-2-F3 primer
ccactagaggagctactgta

SEQ ID NO: 8 - DNA, 18 bp, Artificial Sequence
  SARS-CoV-2-B3 primer
tgacaagctacaacacgt

SEQ ID NO: 9 - DNA, 48 bp, Artificial Sequence
  SARS-CoV-2-FIP primer
aggtgagggttttctacatcactatattggaacaagcaaattctatgg

SEQ ID NO: 10 - DNA, 43 bp, Artificial Sequence
  SARS-CoV-2-BIP primer
atgggttgggattatcctaaatgtgtgcgagcaagaacaagtg

SEQ ID NO: 11 - DNA, 25 bp, Artificial Sequence
  SARS-CoV-2-LoopF primer
cagtttttaacatgttgtgccaacc

SEQ ID NO: 12 - DNA, 21 bp, Artificial Sequence
  SARS-CoV-2-LoopB primer
tagagccatgcctaacatgct

SEQ ID NO: 13 - DNA, 10 bp, Artificial Sequence
  DENV2-BIP Oligonucleotide-Quencher
atctcgtttg

SEQ ID NO: 14 - DNA, 10 bp, Artificial Sequence
  SARS-CoV-2-FIP Oligonucleotide-Quencher
accctcacct

SEQ ID NO: 15 - DNA, 10 bp, Artificial Sequence
  SARS-CoV-2-BIP Oligonucleotide-Quencher
cccaacccat

SEQ ID NO: 16 - DNA, 10 bp, Artificial Sequence
  SARS-CoV-2-LoopF Oligonucleotide-Quencher
ttaaaaactg

SEQ ID NO: 17 - DNA, 10 bp, Artificial Sequence
  SARS-CoV-2-LoopB Oligonucleotide-Quencher
catggctcta

SEQ ID NO: 18 - DNA, 21 bp, Artificial Sequence
  SARS-CoV-2-F3 primer
tccagatgaggatgaagaaga

SEQ ID NO: 19 - DNA, 21 bp, Artificial Sequence
  SARS-CoV-2-B3 primer
agtctgaacaactggtgtaag

SEQ ID NO: 20 - DNA, 41 bp, Artificial Sequence
  SARS-CoV-2-FIP primer
agagcagcagaagtggcacaggtgattgtgaagaagaagag

SEQ ID NO: 21 - DNA, 40 bp, Artificial Sequence
  SARS-CoV-2-BIP primer
tcaacctgaagaagagcaagaactgattgtcctcactgcc

SEQ ID NO: 22 - DNA, 22 bp, Artificial Sequence
  SARS-CoV-2-LoopF primer
ctcatattgagttgatggctca

SEQ ID NO: 23 - DNA, 22 bp, Artificial Sequence
  SARS-CoV-2-LoopB primer
acaaactgttggtcaacaagac

SEQ ID NO: 24 - DNA, 8 bp, Artificial Sequence
  SARS-CoV-2-FIP Oligonucleotide-Quencher
gctgctct

SEQ ID NO: 25 - DNA, 18 bp, Artificial Sequence
  18S_rRNA_F3 primer
gttcaaagcaggcccgag

SEQ ID NO: 26 - DNA, 20 bp, Artificial Sequence
  18S_rRNA_B3 primer
cctccgactttcgttcttga

SEQ ID NO: 27 - DNA, 41 bp, Artificial Sequence
  18S_rRNA_FIP primer
tggcctcagttccgaaaaccaacctggataccgcagctagg

SEQ ID NO: 28 - DNA, 39 bp, Artificial Sequence
  18S_rRNA_BIP primer
ggcattcgtattgcgccgctggcaaatgctttcgctctg

SEQ ID NO: 29 - DNA, 25 bp, Artificial Sequence
  18S_rRNA_LoopF primer
agaaccgcggtcctattccattatt

SEQ ID NO: 30 - DNA, 20 bp, Artificial Sequence
  18S_rRNA_LoopB primer
attccttggaccggcgcaag

SEQ ID NO: 31 - DNA, 8 bp, Artificial Sequence
  18S_rRNA_FIP Oligonucleotide-Quencher
cgaatgcc

(19) United States
(12) Patent Application Publication
(10) Pub. No.: US 2023/0212700 A1
(43) Pub. Date: Jul. 6, 2023

(54) PORTABLE DIAGNOSTIC DEVICE IN THE FORM OF A CYLINDRICAL HOUSING AND USES THEREOF

(71) Applicants: PARIS SCIENCES ET LETTRES, PARIS (FR); ECOLE SUPERIEURE DE PHYSIQUE ET DE CHIMIE INDUSTRIELLES DE LA VILLE DE PARIS, PARIS (FR); CENTRE NATIONAL DE LA RECHERCHE SCIENTIFIQUE, PARIS (FR)

(72) Inventors: Patrick TABELING, PARIS (FR); Pierre GARNERET, PARIS (FR); Elian MARTIN, PARIS (FR); Etienne COZ, MONTROUGE (FR); Elodie BRIENT-LITZLER, VERSAILLES (FR); Jean-Claude MANUGUERRA, PARIS (FR); Jessica VANHOMWEGEN, PARIS (FR)

(73) Assignees: PARIS SCIENCES ET LETTRES, PARIS (FR); ECOLE SUPERIEURE DE PHYSIQUE ET DE CHIMIE INDUSTRIELLES DE LA VILLE DE PARIS, PARIS (FR); CENTRE NATIONAL DE LA RECHERCHE SCIENTIFIQUE, PARIS (FR)

(21) Appl. No.: 17/917,757

(22) PCT Filed: Apr. 9, 2021
(86) PCT No.: PCT/EP2021/059330

§ 371 Date: Oct. 7, 2022

(30) Foreign Application Priority Data
Apr. 9, 2020 (FR) FR2003603

(51) Int. Cl. C12Q 1/70; B01L 3/00

(52) U.S. Cl. CPC C12Q 1/701; B01L 3/5023; B01L 2300/069; B01L 2300/0654; B01L 2300/0681; B01L 2300/0803; C12Q 1/6844

(57) ABSTRACT

A portable diagnostic device for performing the diagnosis of pathogens, such as viruses, bacteria, microorganisms, etc., by rapidly detecting their nucleic acids in a biological sample to be tested. Also, the use of the portable diagnostic device and the methods for detection of at least one nucleic acid sequence of interest implemented with the aid of the portable diagnostic device.

### FIELD OF THE INVENTION

The present invention relates to a portable device for performing the diagnosis of pathogens (viruses, bacteria, microorganisms, etc.) by rapidly detecting their nucleic acids in a biological sample to be tested. The present invention also relates to the uses of the diagnostic device of the invention and to the methods which it enables to implement.

### CONTEXT OF THE INVENTION

Nuclear Acid Amplification Tests (NAATs) detect nucleic acids in the infected sample by amplification. NAATs have the ability to detect pathogens in the early days of infection, well before the development of the immune response. The technique is characterized by high sensitivities (10 - 100 particles) and excellent specificities, due to a primer hybridisation step. Due to its performance, the PCR (Polymerase Chain Reaction)-based NAAT is undoubtedly considered the reference in the field.

Although extremely useful, the disadvantage of PCR-based NAATs is the cost and use of complex equipment. Many commercial instruments are now commercially available (ThermoFisher®, Roche®, Genexpert®, etc.). However, the cost of the equipment varies from €30k to €200k. In many cases, they require highly qualified personnel, and clean, temperature and humidity controlled spaces to perform the tests. Such equipment is located in a limited number of places, hospitals and laboratories, requiring, in most cases, the transport of samples to be analysed, or the transport of patients to reach these places, with significant risks of contamination. Thus, the cost and complexity of PCR-based NAAT hinders the possibility of massive amounts of testing, or limits it to rich countries.

In France, at the beginning of the SARS-CoV-19 epidemic, a few thousand tests were performed each day, a figure to be compared with the tens of thousands of infected people. It took a lot of time and resources to increase the number of tests to 10 - 20,000 per day in April 2020, as the number is still insufficient. In the case of this disease, some countries such as South Korea have implemented a major testing approach, which, coupled with the isolation of positive patients, has resulted in a significant decrease in the spread of the pathogen. However, the deployment of massive testing requires expensive and complex logistics, which cannot be easily deployed in middle-and low-income countries.

In recent years, with the advent of isothermal amplification technologies (Rolling Circle Amplification [RCA], Loop Mediated Amplification [LAMP], Recombinase Polymerase Amplification [RPA], etc.), which do not require thermocycling to amplify nucleic acids, a new field of research has opened up, raising the hope of performing NAATs on site, using much simpler and less expensive equipment.), which do not require thermocycling to amplify nucleic acids, a new field of research has opened up, raising hopes of performing NAATs on site, using equipment that is much simpler and less expensive than PCR platforms. Today, there are about twenty isothermal amplification techniques (A. Niemz, T. Ferguson, D. Boyle. “Point of care nucleic acid testing for infectious diseases”, Trends in biotechnology, 29, 240, 2011.). Among them, the LAMP isothermal amplification, which operates at 65° C., should be highlighted because of its performance, in terms of amplification rates (twice as fast as PCR), sensitivity and specificity (similar to PCR), as well as the large amounts of cDNA (Complementary Deoxyribonucleic Acid) produced during the reaction, facilitating reading.

Recently, NAATs based on RPA and LAMP have been performed, mostly inspired by ideas developed in recent years in the field of “paper microfluidics” (S. Vella et al, “Measuring markers of liver function using a micropatterned paper device designed for blood from a fingerstick”, Analytical Chemistry, vol. 84, pp. 2883-2891, 2012; J. Linnes et al, “Paper based molecular diagnostic for Chlamydia trachomatis, RSC Advances, vol. 4, pp. 42245-42251, 2014; L. Magro et al, “Paper microfluidics for nucleic acid amplification testing (NAAT) of infectious diseases”, Lab on a Chip, 14, 2347-2371, 2017; L. Magro et al, “Paper-based RNA detection and multiplexed analysis for Ebola virus diagnostics”, Scientific Reports 7, 1347, 2017). Without degradation of quality, these tests replace the PCR technique in terms of simplicity, compactness and cost, making it portable for use in medical practices. However, in practice, the existing types of tests do not allow for the development of these “point of care” or “field” uses, for various reasons (no freeze-drying of products, no RNA extraction, complicated system to use for medical staff, too complex surrounding instrumentation, etc.).

### BRIEF OVERVIEW OF THE INVENTION

In response to current (i.e. COVID-19 pandemic) and future health needs, the inventors have developed a new portable and easily deployable large-scale diagnostic device for the diagnosis of pathogens (viruses, bacteria, microorganisms, etc.) based on nucleic acid detection. This new device allows for rapid, reliable and safe testing, and can also be used to test one or more samples. Moreover, this new device, which can be single-use and/or disposable, has the advantage of being inexpensive and locally usable. Thus and thanks to the diagnostic device of the invention, diagnoses could be carried out at the doctor’s office, in the hospital emergency department, in the workplace, in pharmacies or even at home.

A first object of the invention is therefore a portable diagnostic device in the form of a cylindrical housing. A second object of the invention concerns the various possible uses of the diagnostic device. A further object of the invention relates to methods that can be easily carried out by a user, which allow the rapid diagnosis of pathogens (viruses, bacteria, microorganisms, etc.) in a reliable and safe manner. Another object of the invention also relates to methods for manufacturing the diagnostic device of the invention.

### LIST OF FIGURES

The following figures illustrate the invention, without limiting its scope.

FIG. 1 shows a perspective view of the diagnostic device of the invention. (1) diagnostic device; (3) upper disc; (5) lower disc; (8) membrane; (12) reaction zone and (13a and 13b) pastilles.

FIG. 2 shows a schematic top view of the diagnostic device of the invention. (3) upper disc; (8) membrane; (9) window and (13a and 13b) pastilles.

FIG. 3 shows a schematic bottom view of the diagnostic device of the invention when it comprises a recess. (5) lower disc; (6) recess and (13a and 13b) pastilles.

FIG. 4 shows an exploded perspective view of the diagnostic device of the invention shown in FIGS. 1, 2 and 3. (1) diagnostic device; (3) upper disc; (5) lower disc; (7) receiving opening; (8) membrane; (9) window; (11) optical filter; (13a and 13b) pastilles; and (15) absorbing material.

FIG. 5 shows a vertical cross-sectional view of the diagnostic device of the invention along the V-V line (A) of FIG. 2 (i.e. without recess at the lower disc (5)) and (B) of FIG. 3 (i.e. with recess at the lower disc (5)). (1) diagnostic device; (3) upper disc; (5) lower disc; (6) recess; (7) receiving opening; (8) membrane; (9) window; (10) sealing material; (11) optical filters; (13b) pastille; and (15) absorbing material.

FIG. 6 shows a top perspective view of a first alternative implementation of the diagnostic device of the invention allowing eight tests to be performed on the same device. (2) diagnostic block (black triangle); (3) upper disc; (4) detector unit (white arc); (7) receiving opening and (9a and 9b) windows.

FIG. 7 shows a top perspective view of a second alternative implementation of the diagnostic device of the invention allowing twelve tests to be performed on the same device. (3) upper disc; (8) membranes; (12a, 12b) reaction zones and (13a and 13b) pastilles.

FIG. 8 shows a bottom view of the upper disc of the diagnostic device of the invention, which comprises three-dimensional elements for assisting the rotational movement. (3) upper disc; (8) diaphragms; (11) optical filter; (16) groove; (17) skirt and (18) slide.

FIG. 9 shows a schematic view of the plans for printing the upper disc of the diagnostic device of the invention using a 3D printer. The figures and numbers correspond to dimensions in millimetres (mm).

FIG. 10 shows a schematic view of the plans for printing the lower disc of the diagnostic device of the invention using a 3D printer. The figures and numbers correspond to dimensions in millimetres (mm).

FIG. 11 shows the experimental data obtained in Example 2. Devices used for the detection of DENV2 and SARS-CoV-2 RNA in biological samples. (a) Device for the detection of DENV2, sample containing DENV2 RNA (positive sample). Black bar: fluorescence intensity of the internal control measured at t =45 minutes after subtraction of the initial fluorescence. Grey hatched bar: fluorescence intensity of the RT-LAMP DENV2 pastille measured at t = 45 min after subtraction of the initial fluorescence. (b) Device for the detection of DENV2, negative sample. Black bar: fluorescence intensity of the internal control measured at t =45 min after subtraction of the initial fluorescence. White bar: fluorescence intensity of the pastille RT-LAMP DENV2 measured at t =45 min after subtraction of the initial fluorescence. (c) Image of DENV2 pastilles, positive sample on the left, negative on the right. Photograph taken with a smartphone. (d) Device for detection of SARS-CoV-2, sample containing SARS-CoV-2 RNA (positive sample). Black bar: fluorescence intensity of the internal control measured at t =45 minutes after subtraction of the initial fluorescence. Grey hatched bar: fluorescence intensity of the RT-LAMP SARS-CoV-2 pastille measured at t =45 min after subtraction of the initial fluorescence. (e) Device for the detection of DENV2, negative sample. Black bar: fluorescence intensity of the internal control measured at t =45 min after subtraction of the initial fluorescence. White bar: fluorescence intensity of the RT-LAMP SARS-CoV-2 pastille measured at t =45 min after subtraction of the initial fluorescence. (f) Images of the SARS-CoV-2 pastilles, positive sample on the left, negative on the right. Photograph taken with a digital camera.

FIG. 12 shows a schematic view of the dispenser in its closed configuration. (20) dispenser; (23) base; (24) cover and (90) actuator.

FIG. 13 shows a schematic view of the dispenser in its open configuration. (20) dispenser; (23) base; (24) cover; (25) circular cavity; (31) diagnostic device and (68) rotating disc.

FIG. 14 shows a schematic exploded view of the diagnostic device of the invention. (31) diagnostic device; (33) upper disc; (35) lower disc; (43a and 43b) reagent pastilles; (45) absorbent material; (50) cavity; (51) circular slot; (52a) opening; (53) radial slot; (54) central hole; (62) capsule; (62a) part of the capsule suitable for insertion into the diagnostic device; (62b) cover; r3 axis; r′3 axis and yy′ axis

FIG. 15 shows a schematic view of the upper disc (33) with two openings. (33) diagnostic device; (50) cavity; (51) circular slot; (52a and 52b) receiving openings; (53) radial slot; (55) central cleat; width a; axis r1; axis r3; angles α and β.

FIG. 16 shows a schematic view of the upper disc (33) with an opening. (33) diagnostic device; (37) receiving opening; (38) capture membrane; r1 axis.

FIG. 17 shows a schematic view of the interior of the lower disc (35). (35) lower disc; (54) central hole; (56) radial slot; (57) gripping means; (58) radial window; (59) small tank; (60) tenon; width a′; axis r′1; axis r′2; axis r′3 and angle /2.

FIG. 18 shows a schematic view of the capsule in its open configuration. (43a and 43b) test pastilles; (62) capsule; (62a) part of the capsule adapted to be inserted into the diagnostic device; (62b) cover; (62c) link; (64a, 64b and 64c) hollow bosses and (66a, 66b and 66c) hollow bosses.

FIG. 19 shows a schematic view of the lower disc (35) into which the capsule (62), in its open configuration, is inserted. (35) lower disc; (58) radial window; (59) small tank; (60) tenon; (62) capsule, r′2 axis and r′3 axis.

FIG. 20 shows the arrangement of the upper disc (33) in relation to the lower disc (35) when the diagnostic device (31) is in position 1. (33) upper disc; (35) lower disc; (52a and 52b) receiving openings; (58) radial window; (59) small tank; r1 axis; r2 axis; r3 axis; r′1 axis; r′2 axis and r′3 axis.

FIG. 21 shows the arrangement of the upper disc (33) with respect to the lower disc (35) when the diagnostic device (31) is in the intermediate position. (33) upper disc; (35) lower disc; (52a and 52b) receiving openings; (58) radial window; r1-axis; r2-axis; r3-axis; r′1-axis; r′2-axis and r′3-axis.

FIG. 22 shows the arrangement of the upper disc (33) in relation to the lower disc (35) when the diagnostic device (31) is in position 2. (33) upper disc; (35) lower disc; (52a and 52b) receiving openings; (56) radial slot; r1-axis; r2-axis; r3-axis; r′1-axis; r′2-axis and r′3-axis.

FIG. 23 shows schematically one type of arrangement implemented in a dispenser (20). (20) dispenser; (23) base; (24) cover; (31) diagnostic device; (70) power supply; (72) control electronics; (74) power stage; (76) first metering pump; (78) ethanol tank; (80) nozzle; (82) second metering pump; (84) eluent tank; (86a and 86b) nozzles and (88) motor.

FIG. 24 shows a schematic view of the top of the detector (21) in its open configuration. (62) capsule; (91) sensor body; (92) cover and (93) plate.

FIG. 25 shows a schematic view of the top of the detector (21) in its closed configuration. (62) capsule; (89) cell; (91) detector body and (93) plate.

FIG. 26 shows schematically one type of arrangement implemented in a detector (21). (94) power supply; (95) control electronics; (96) power stage; (97) heating resistors and (98) diodes.

FIG. 27 shows a schematic diagram of the serial operation with n capsules of the diagnostic device of the invention in combination with a detection system comprising the joint use of a dispenser and a detector.

FIG. 28 shows the experimental data obtained in Example 3. Detection of SARS-CoV 2 RNA in artificial saliva and nasopharyngeal fluid samples (panel provided by the X Prize Foundation in the XPrize rapid covid testing competition). A. Fluorescent signal on amplification pastilles detecting SARS-CoV-2, for nasopharyngeal samples extracted by the device of the invention. The detection limit is around 5 genomic copies/µL of sample. B. Fluorescent signal on amplification pastilles detecting SARS-CoV-2, for salivary samples extracted by the device of the invention. The detection limit is around 5 genomic copies/µL of sample.

### DETAILED DESCRIPTION

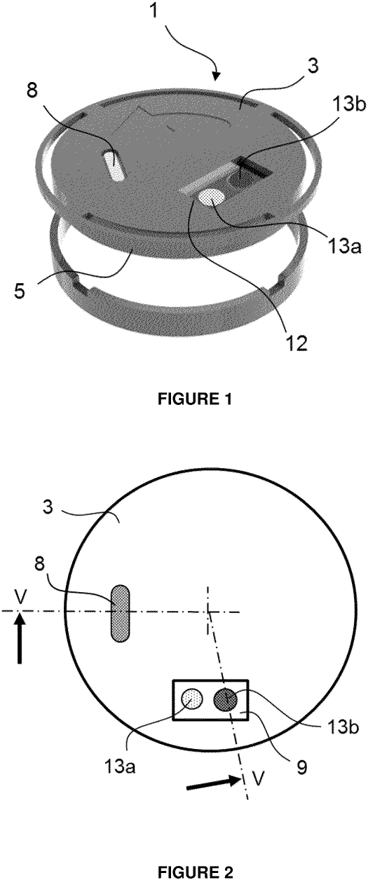

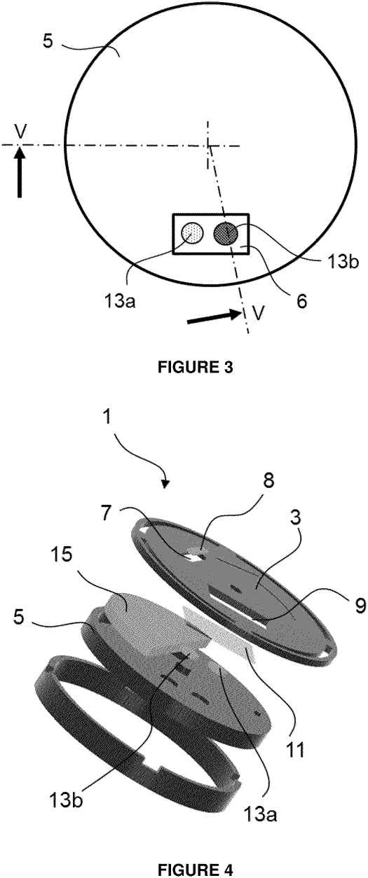

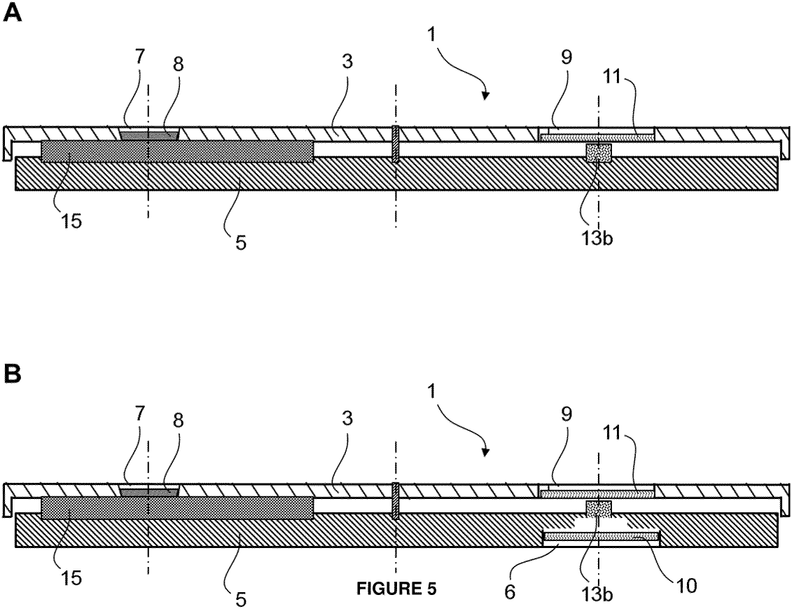

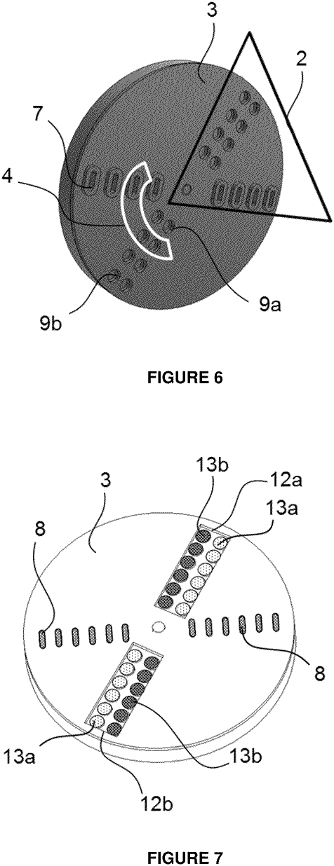

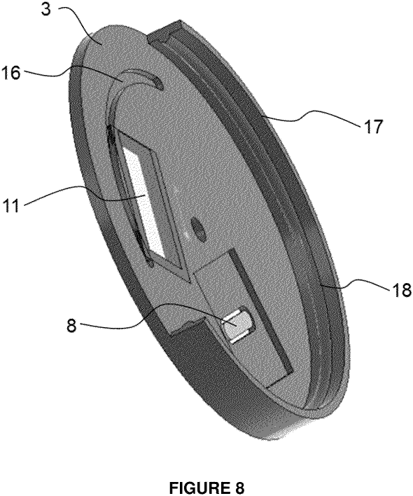

The invention involves coupling an extraction unit and an amplification/detection unit in a portable device, which comprises lyophilized reagents in situ. Examples of this device and its variants are shown in FIGS. 1 to 10 and 12 to 27.

A first aspect of the invention relates to a portable diagnostic device in the form of a cylindrical housing including means:
extraction of nucleic acids (DNA and/or RNA) from a test sample,
isothermal amplification (e.g. (RT)-LAMP, (RT)-LAMP-QUASR [Quenching of Unincorporated Amplification Signal Reporters]) of at least one nucleic acid sequence of interest likely to be present in the test sample, and
detection of amplicons if the at least one nucleic acid sequence of interest is present in the test sample.

A first very general embodiment of the invention according to this first aspect relates to a portable diagnostic device (1) in the form of a cylindrical housing comprising two superimposed coaxial discs (3, 5) mounted for rotation with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced at an angle of at least 30° on centre,
a lower disc (5) comprising:
   an absorbent material (15) capable of containing a volume of liquid, and
   at least one reaction zone (12) comprising at least one, preferably at least two, independent, adjacent, biologically compatible pastilles (13a, 13b) capable of allowing the amplification and detection of at least one nucleic acid sequence of interest likely to be present in the abovementioned biological sample in the presence of appropriate reagents,
the receiving opening (7), the window (9), the absorbent material (15) and the reaction zone (12) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
at a second position in which, for this detection unit (4), its receiving opening (7) is in line with its reaction zone (12) and covers it in part or in full so as to allow the elution of the abovementioned nucleic acids, and
to position itself, by a rotation, on the above-mentioned first position, so that, for this detection unit (4), its window (9) is in line with its reaction zone (12), so as to allow the amplification and the detection of the above-mentioned at least one nucleic acid sequence of interest likely to be present in the biological sample
As described, the transition from the above-mentioned second position to the above-mentioned first position can therefore be made either by a rotation which is the reverse of that allowing the transition from the above-mentioned first position to the above-mentioned second position, or by a rotation in the same direction as the latter. Advantageously, this is done by a reverse rotation, which allows the return to the initial position to be made more directly, by a shorter path, avoiding the observation window (9) approaching the absorbent material (15). The invention then relates to the diagnostic device (1) as described above in which the passage from said second position to said first position is made by a rotation opposite to that allowing the passage from said first position to said second position. In other words, a very general advantageous embodiment of the invention concerns a portable diagnostic device (1) in the form of a cylindrical housing comprising two superimposed coaxial disks (3, 5) mounted for rotation with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced apart by a centre angle of at least 30°, in particular by a centre angle of between 30° and 320°,
a lower disc (5) comprising:
   an absorbent material (15) capable of containing a volume of liquid, and
   at least one reaction zone (12) comprising at least one, preferably at least two, independent, adjacent, biologically compatible pastilles (13a, 13b) capable of allowing the amplification and detection of at least one nucleic acid sequence of interest likely to be present in the abovementioned biological sample in the presence of appropriate reagents,
the receiving opening (7), the window (9), the absorbent material (15) and the reaction zone (12) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
at a second position in which, for this detection unit (4), its receiving opening (7) is located in line with its reaction zone (12) and covers it in part or in full so as to allow the elution of the abovementioned nucleic acids, the arrangement of the absorbent material (15) being such that, during rotation, the window (9) cannot come into line and contact with it, and
return, by a reverse rotation, to the above-mentioned first position, so that, for this detection unit (4), its window (9) is in line with its reaction zone (12), so as to allow the amplification and detection of the above-mentioned at least one nucleic acid sequence of interest likely to be present in the biological sample, the arrangement of the absorbent material (15) being such that, during reverse rotation, the window (9) cannot come into line and contact with it.
Another very general embodiment of the invention concerns the diagnostic device (1) as described above in which the lower disc (5) further comprises at least one recess (6) which is either equipped with a sealing material (10) integrated into the mass of the lower disc, or is capable of receiving a sealing material (10) which can be positioned on said recess in a removable manner, and which is positioned in line with and integral with said at least one reaction zone (12), and in particular allowing light to reach said at least one preferably at least two biologically compatible pastilles (13a and 13b) of said at least one reaction zone (12). In other words, this other general embodiment relates to the portable diagnostic device (1) in the form of a cylindrical housing as described above comprising two superimposed coaxial disks (3, 5) rotatably mounted with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced at an angle of at least 30° on centre,
a lower disc (5) comprising:
   an absorbent material (15) capable of holding a volume of liquid,
   at least one reaction zone (12) comprising at least one, preferably at least two, independent, adjacent, biologically compatible pastilles (13a, 13b) capable of allowing the amplification and detection of at least one nucleic acid sequence of interest likely to be present in the above-mentioned biological sample in the presence of appropriate reagents, and
   at least one recess (6) which is either equipped with a sealing material (10) integrated in the mass of the lower disc or is able to receive a sealing material (10) which can be positioned on said recess in a removable way, and which is positioned in line with and integral with said at least one reaction zone (12), and in particular allowing light to reach said at least one preferably at least two biologically compatible pastilles (13a and 13b) of said at least one reaction zone (12);
the receiving opening (7), the window (9), the absorbent material (15), the reaction zone (12) and the recess (6) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
at a second position in which, for this detection unit (4), its receiving opening (7) is in line with its reaction zone (12) and covers it in part or in full so as to allow the elution of the abovementioned nucleic acids, and
to position itself, by a rotation, on the above-mentioned first position, so that, for this detection unit (4), its window (9) is in line with its reaction zone (12), so as to allow the amplification and the detection of the above-mentioned at least one nucleic acid sequence of interest likely to be present in the biological sample

In the same way, the passage from the above-mentioned second position to the above-mentioned first position can thus be made either by a rotation opposite to that allowing the passage from the above-mentioned first position to the above-mentioned second position, or by a rotation in the same direction as the latter. Advantageously, this other general embodiment concerns the portable diagnostic device (1) in the form of a cylindrical housing as described above comprising two superimposed coaxial discs (3, 5) mounted so as to rotate with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced apart by a centre angle of at least 30°, in particular by a centre angle of between 30° and 320°,
a lower disc (5) comprising:
   an absorbent material (15) capable of holding a volume of liquid,
   at least one reaction zone (12) comprising at least one, preferably at least two, independent, adjacent, biologically compatible pastilles (13a, 13b) capable of allowing the amplification and detection of at least one nucleic acid sequence of interest likely to be present in the above-mentioned biological sample in the presence of appropriate reagents, and
   at least one recess (6) which is either equipped with a sealing material (10) integrated in the mass of the lower disc or is able to receive a sealing material (10) which can be positioned on said recess in a removable way, and which is positioned in line with and integral with said at least one reaction zone (12), and in particular allowing light to reach said at least one preferably at least two biologically compatible pastilles (13a and 13b) of said at least one reaction zone (12);
the receiving opening (7), the window (9), the absorbent material (15), the reaction zone (12) and the recess (6) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
to a second position in which, for this detection unit (4), its receiving opening (7) is located in line with its reaction zone (12) and covers it in part or in full so as to allow the elution of the abovementioned nucleic acids, the arrangement of the absorbent material (15) being such that, during rotation, the window (9) cannot come into line and contact with it, and
return, by a reverse rotation, to the above-mentioned first position, so that, for this detection unit (4), its window (9) is in line with its reaction zone (12), so as to allow the amplification and detection of the above-mentioned at least one nucleic acid sequence of interest likely to be present in the biological sample, the arrangement of the absorbent material (15) being such that, during reverse rotation, the window (9) cannot come into line and contact with it.
By “a portable diagnostic device (1) in the form of a cylindrical housing” is meant a device of approximately hand size (e.g. 4.00 cm to 20.00 cm in diameter, i.e. 4, 5, 6, 7, 8, 9, 10, 11, 12, 13, 14, 15, 16, 17, 18, 19 or 20 cm) which is easy to grip and ergonomic. Due to the materials chosen, it is inexpensive to produce. It is also light, which makes it easy to use. Moreover, due to its size, it is also easy to transport from one room to another, e.g. from a laboratory or a hospital. According to another embodiment, the invention thus relates to the diagnostic device (1) as described above in which the upper (3) and lower (5) discs are made of a material selected from: polylactic acid (PLA) and polycarbonate (CO—O—pPh—C(CH₃)₂ —pPh—O)ₙ ). In particular, it should be noted that the materials used to manufacture these discs can be opaque or transparent (i.e. light transmitting). According to another embodiment, the invention also relates to the diagnostic device (1) as described above, the diameter to height ratio of the device being between 5 and 30. In particular, the diameter to height ratio of the device is from 5 to 10, from 10 to 15, from 15 to 20, from 20 to 25, from 25 to 30, from 10 to 20 or from 15 to 25. In particular, the diameter to height ratio of the device is 5, 6, 7, 8, 9, 10, 11, 12, 13, 14, 15, 16, 17, 18, 19, 20, 21, 22, 23, 24, 25, 26, 27, 28, 29 or 30.

Advantageously, the diagnostic device (1) of the invention also comprises three-dimensional structures that help guide the rotations of the upper disc on the lower disc and vice versa, thus making it more ergonomic and even easier to use. According to another embodiment, the invention thus relates to the diagnostic device (1) as described above in which said rotations are guided by a system of groove(s) (16) and pin(s), also called cleat(s).

Alternatively, the diameter of the upper (3) and lower (5) discs can be different. The system of groove(s) and pin(s) may then be replaced or supplemented by a system of skirt(s) (17) and slide(s) (18). According to this embodiment, the invention thus relates to the diagnostic device (1) as described above in which the upper disc (3) is larger than the lower disc (5) or in which the lower disc (5) is larger than the upper disc (3). In particular, it should be noted that the skirt(s) are arranged on the larger disc and the slide(s) (18) on the upper (3) and lower (5) discs to guide the rotations of the device. Therefore, according to another embodiment, the invention relates to the diagnostic device (1) as described above in which said rotations are guided by a system of skirt(s) (17) and slide(s) (18).

Advantageously, the diagnostic device (1) of the invention also comprises three-dimensional structures which assist in the assembly of the upper (3) and lower (5) discs to each other. For example, the diagnostic device (1) of the invention comprises a stud at the centre of the lower disc (5), or the upper disc (3), which fits into a circular recess cut out in the centre of the upper disc (3), or the lower disc (5). It may also be a ring (i.e. an additional part) on which the upper (3) and lower (5) discs fit, said ring comprising means allowing the discs to rotate on each other (e.g. slides, grooves, pins or cleats, etc.).

Advantageously, the diagnostic device (1) of the invention also comprises parts, moulded or not with (on) the discs, allowing the introduction of the biological sample to be tested and the reagents (or buffers) without loss of liquid.

Biological test sample” means any type of biological sample that may contain nucleic acids. This may include, but is not limited to, blood, urine, sweat, saliva, ENT secretions (e.g. nose, ears, throat), cerebrospinal fluid, lymphatic fluid, amniotic fluid, etc. It may also include nasopharyngeal swabs, cervico-vaginal swabs, etc. The biological sample to be tested may also come from a mammal. In particular, the sample is from a mammal selected from: humans (i.e. children, adults, women and men), monkeys, felines, canines, equines, cervids, cattle, sheep and poultry. Preferably, the sample should be human.

By “nucleic acids” is meant deoxyribonucleic acid (DNA) and/or ribonucleic acid (RNA). The invention, due to its circular configuration, also advantageously allows the elution of nucleic acids, and the amplification and detection of at least one nucleic acid sequence (DNA and/or RNA) of interest likely to be present in a biological sample at the same place in the device, thus avoiding too many manipulations. Similarly, the invention is configured in such a way that the window (9) equipped with an optical filter (11) can never be in line with and in contact with the absorbent material (15), known as the capillary buffer, thus avoiding contamination of the reaction zone (12) by the absorbents caught in the absorbent material (15).

Nucleic acid sequence of interest’ means a nucleic acid sequence present in the genome of a pathogen (virus, bacterium, microorganism, etc.) and whose detection in a biological sample allows the diagnosis of a pathology. It may therefore be, for example, a DNA sequence contained in the genomic plasmid of a bacterium (e.g. Salmonella, Pseudomonas, Staphylococcus, Escherichia, Streptococcus, Helicobacter, etc.) or an RNA sequence contained in the transcriptome of that bacterium. It may also be a DNA and/or RNA sequence contained in the genome of a virus (e.g. SARS-CoV-2, Dengue virus, human immunodeficiency virus [HIV], etc.)). As the invention is a diagnostic device, it makes it possible to test a biological sample to find out whether it contains, for example, an infection (viral, bacterial or other microorganism, etc.), hence the expression “likely to be present in the above-mentioned biological sample”. In other words, in the current health context (i.e. COVID-19 pandemic), the diagnostic device of the invention makes it possible to test a patient, or even the population, to find out if he or she has (or does not have) an infection (i.e. SARS-CoV-2 viral infection) and to take the appropriate measures (medical treatment, confinement, etc.). By “nucleic acid sequence of interest” is also meant a nucleic acid sequence present in the genome (DNA) or transcriptome (RNA) of a mammal (e.g. man), the presence of which would be linked to a predisposition to a pathology or to the diagnosis of a pathology. The diagnostic device of the invention as described thus offers the advantage of being versatile and easily adaptable to the diagnosis sought and/or the health context.

Detection unit’ means the minimum set of equipment that enables the diagnosis of a biological sample to be carried out. As indicated, it is provided with:
a biologically compatible membrane (8), which receives the biological sample to be tested, and which allows nucleic acids (DNA and/or RNA) to be extracted from the sample to be tested and to be retained by affinity in the presence of the appropriate reagents when it is in line with the absorbent material;
of an absorbent material (15), which collects and stores the liquids that pass through said membrane (8);
a reaction zone (12) comprising at least one, preferably at least two, biologically compatible pastilles (13a and 13b) capable of allowing, after elution of the nucleic acids (DNA and/or RNA) from the abovementioned membrane (8), the amplification and detection of at least one nucleic acid sequence of interest likely to be present in the biological sample in the presence of appropriate reagents;
a window (9) equipped with an optical filter (11) on the upper disc (3) and capable of being positioned in line with the reaction zone (12); and
possibly a recess (6) equipped with a sealing material (10) on the lower disc (5) below the reaction zone (12) and in particular allowing light to reach the at least one, preferably at least two pastilles (13a and 13b).
By “window” is meant an opening cut in the material of the upper disc (3). However, in order to ensure the hermeticity and watertightness of the diagnostic device (1) of the invention, the window or windows are provided with an optical filter (11). This optical filter may be removably positioned (e.g. adhesive film, flap, shutter etc.) or integrated into the mass of the upper disc (3), or even placed on an external device. By “optical filter” is meant a device which allows all or part of the light radiation to pass through and which enables the light beam at the test outlet to be analysed. Thus, by “optical filter” we mean materials with specific optical properties (e.g. bandpass, notch, high-pass, low-pass), colored filters (e.g. photographic gelatins, etc.), devices such as optical cubes allowing the reading of a fluorescent signal. Furthermore, and in the context of a colorimetric test or a turbidimetric test (or any other amplification visualization method), it may be a filter assisting in the reading of the test. In particular, the invention relates to the diagnostic device (1) as described above in which the optical filter is selected from: a bandpass filter, a notch filter, a high-pass filter, a low-pass filter, photographic gelatin and an optical cube.

By “recess” is meant an opening cut in the material of the lower disc (5). However, in order to ensure the hermeticity and sealing of the diagnostic device (1) of the invention, the recess(es) are provided with a sealing material (10). This sealing material may be removably positioned (e.g. adhesive film, flap, shutter etc.) or integrated into the mass of the lower disc (5), or even placed on an external device. Sealed material” means any type of plastic or other material that is impermeable and compatible with amplification reactions. It may be transparent and allow light to pass through for transmission fluorescence reading. It can also be opaque for colorimetric reading or fluorescence reading by reflection. The invention thus relates to the diagnostic device (1) as described above in which the sealing material is selected from: a bandpass filter, a notch filter, a high-pass filter, a low-pass filter, photographic gelatin and an optical cube.

Absorbent material”, also known as capillary buffer or absorbent sponge, means a material which, upon extraction of the nucleic acids contained in the test sample at the membrane (8) in the presence of the appropriate reagents [also known as buffers], is capable of absorbing, containing and storing liquids which pass through the membrane (8). This material, capable of holding a volume of at least 1 mL (1 cm³), can furthermore be placed on a receptacle provided to accommodate it. This absorbent material may be, in particular, an absorbent material made of cellulose, glass fibre or cotton flock, any other absorbent material or, for example, an absorbent material that can be used for a conventional immuno-chromatographic test. According to another embodiment, the invention thus relates to the diagnostic device (1) as described above in which the absorbent material (15) is disposed in a receptacle which is positioned either on the upper surface of the lower disc or integrated into the mass of the latter. According to another embodiment, the invention also relates to the diagnostic device (1) as described above wherein the absorbent material (15) is capable of containing a volume of at least 1 mL (1 cm³). For the purposes of the invention, “at least 1 mL” means a volume of from 1 mL to 2 mL or from 1 mL to 1.50 mL. “At least 1 mL” also means that the volume absorbable by the absorbent material (15) is 1 mL; 1.10 mL; 1.15 mL; 1.20 mL; 1.25 mL; 1.30 mL; 1.35 mL; 1.40 mL; 1.45 mL; 1.50 mL; 1.55 mL; 1.60 mL; 1.65 mL; 1.70 mL; 1.75 mL; 1.80 mL; 1.85 mL; 1.90 mL; 1.95 mL; or 2.00 mL. In particular, the invention relates to the diagnostic device (1) as described above wherein the absorbent material (15) is arranged in a receptacle which is positioned either on the upper surface of the lower disc or integrated into the mass of the latter, and said absorbent material (15) is preferably able to contain a volume of at least 1 mL. In particular, the invention also relates to the diagnostic device (1) as described above wherein the absorbent material (15) is selected from: a cellulose absorbent material, glass fibre or cotton flock and an absorbent material suitable for use in an immuno-chromatographic test.

By “biologically compatible membrane capable of allowing the extraction and retention of nucleic acids ... in the presence of appropriate reagents”, also called capture membrane, is meant a membrane capable of receiving a biological sample to be tested, of supporting the addition of reagents (or buffers) allowing the extraction of the nucleic acids (DNA and/or RNA) contained in the said sample, and of having physico-chemical properties capable of retaining (e.g. by affinity) the extracted nucleic acids prior to their elution. This membrane may in particular be made of cellulose, silica, fibreglass, or any other porous material allowing the physicochemical retention of the nucleic acids. According to another embodiment, the invention thus concerns the diagnostic device (1) as described above in which the biologically compatible membrane (8) capable of allowing the extraction and retention of nucleic acids is made of a material chosen from: cellulose, silica and glass fibre.

By “Biologically compatible pastilles ... capable of allowing the amplification and detection of at least one nucleic acid sequence of interest ... in the presence of appropriate reagents” is meant pastilles made of a material capable of collecting the nucleic acids (DNA and/or RNA) extracted from the biological sample to be tested after their elution from the membrane (8). This material, permeable to light, also allows, in the presence of appropriate reagents (e.g. primers, fluorophores, enzymes, dNTPs [any deoxyribonucleoside triphosphate], etc.), to support and ensure the amplification and then the visual detection (fluorescence, colorimetry, etc.) of at least one nucleic acid sequence likely to be present in the biological sample to be tested. These pastilles may in particular be made of glass fibre (with or without an adjuvant binder), of cellulose, or of any other porous material that is biocompatible or made biocompatible by a prior treatment. It should also be noted that these pastilles are adjacent, i.e. side by side, while being independent, i.e. they do not touch each other thus avoiding contamination between adjacent pastilles. Furthermore, these pastilles per detection unit are at least one, preferably at least two, i.e. a detection unit may comprise one, two, three, four, five or six pastilles. According to another embodiment, the invention thus relates to the diagnostic device (1) as described above in which the at least two biologically compatible amplification pastilles (13a, 13b) are made of a material selected from: glass fibre (whether or not containing a binder adjuvant) and cellulose.

Advantageously, these suitable reagents for the amplification and detection of at least one nucleic acid sequence (DNA and/or RNA) can be lyophilized in situ on the surface of (or in) the at least one, preferably at least two, biologically compatible pastilles (13a and 13b) of the reaction zone (12). As freeze-drying is one of the best ways to maintain the integrity of these reagents in the long term, it is possible, after manufacturing the diagnostic device (1) of the invention, to freeze-dry these reagents therein. The diagnostic device (1) of the invention can then be prepared in advance on a large scale, stored on a long-term basis and deployed rapidly in the event of a health crisis. It can also be used immediately after the freeze-drying step described above.

Advantageously, the invention thus concerns the diagnostic device (1) as described above in which at least one reaction zone (12) comprises at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in said biological sample.

In other words, the invention also relates to the portable diagnostic device (1) in the form of a cylindrical housing as described above comprising two superimposed coaxial discs (3, 5) mounted for rotation with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced at an angle of at least 30° on centre,
a lower disc (5) comprising:
   an absorbent material (15) capable of containing a volume of liquid,
   at least one reaction zone (12) comprising at least one, preferably at least two, biologically compatible, adjacent and independent amplification pastilles (13a, 13b), at least one of which contains lyophilized reagents capable of allowing the amplification and detection of at least one nucleic acid sequence of interest likely to be present in the above-mentioned biological sample, and
   optionally at least one recess (6) which is either equipped with a sealing material (10) integrated in the mass of the lower disc or is able to receive a sealing material (10) which can be positioned on said recess in a removable way, and which is positioned in line with and integral with said at least one reaction zone (12), and in particular allowing light to reach said at least one preferably at least two biologically compatible pastilles (13a and 13b) of said at least one reaction zone (12);
the receiving opening (7), the window (9), the absorbent material (15), the reaction zone and possibly the recess (6) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
at a second position in which, for this detection unit (4), its receiving opening (7) is in line with its reaction zone (12) and covers it in part or in full so as to allow the elution of the abovementioned nucleic acids, and
to position itself, by a rotation, on the above-mentioned first position, so that, for this detection unit (4), its window (9) is in line with its reaction zone (12), so as to allow the amplification and the detection of the above-mentioned at least one nucleic acid sequence of interest likely to be present in the biological sample

In the same way, the passage from the above-mentioned second position to the above-mentioned first position can thus be made either by a rotation opposite to that allowing the passage from the above-mentioned first position to the above-mentioned second position, or by a rotation in the same direction as the latter. Advantageously, the invention also relates to the portable diagnostic device (1) in the form of a cylindrical housing as described above comprising two superimposed coaxial discs (3, 5) mounted for rotation with respect to each other and defining a closed volume, namely:
an upper disc (3) comprising:
   at least one receiving opening (7) of a biological sample to be tested which is provided with a biologically compatible membrane (8) and capable of allowing the extraction and retention of nucleic acids from this biological sample in the presence of appropriate reagents, and
   at least one window (9) which is either equipped with an optical filter (11) integrated in the mass of the upper disc or is able to receive an optical filter (11) which can be positioned on said window in a removable manner, said receiving opening (7) and said window (9) being angularly spaced apart by a centre angle of at least 30°, in particular by a centre angle of between 30° and 320°,
a lower disc (5) comprising:
   an absorbent material (15) capable of containing a volume of liquid,
   at least one reaction zone (12) comprising at least one, preferably at least two, biologically compatible, adjacent and independent amplification pastilles (13a, 13b), at least one of which contains lyophilized reagents capable of allowing the amplification and detection of at least one nucleic acid sequence of interest likely to be present in the above-mentioned biological sample, and
   optionally at least one recess (6) which is either equipped with a sealing material (10) integrated in the mass of the lower disc or is able to receive a sealing material (10) which can be positioned on said recess in a removable way, and which is positioned in line with and integral with said at least one reaction zone (12), and in particular allowing light to reach said at least one preferably at least two biologically compatible pastilles (13a and 13b) of said at least one reaction zone (12);
the receiving opening (7), the window (9), the absorbent material (15), the reaction zone and possibly the recess (6) form a detection unit (4) such that, by rotation, the upper disc can successively
from a first position in which, for this detection unit (4), its receiving opening (7) is in line with its absorbent material (15), so as to allow the extraction and retention of said nucleic acids, and its window (9) is in line with its reaction zone (12),
at a second position in which, for this detection unit (4), its receiving opening (7) is located in line with its reaction zone (12) and covers it in part or in full so as to allow the elution of the abovementioned nucleic acids, the arrangement of the absorbent material (15) being such that, during rotation, the window (9) cannot come into line and contact with it, and
return, by a reverse rotation, to the above-mentioned first position, so that, for this detection unit (4), its window (9) is in line with its reaction zone (12), so as to allow the amplification and detection of the above-mentioned at least one nucleic acid sequence of interest likely to be present in the biological sample, the arrangement of the absorbent material (15) being such that, during reverse rotation, the window (9) cannot come into line and contact with it.

In particular, this embodiment may include:
the diagnostic device (1) as described above wherein the reaction zone (12) comprises two pastilles, one (13a or 13b) serving as a test pastille and the other (13b or 13a) serving as a negative control pastille; and/or
the diagnostic device (1) as described above wherein the reaction zone (12) comprises two pastilles, one (13a or 13b) serving as a test pastille and the other (13b or 13a) serving as a positive control pastille; and/or
the diagnostic device (1) as described above wherein the reaction zone (12) comprises three pastilles, one serving as a test pastille and the other two serving as negative and positive control pastilles; and/or
the diagnostic device (1) as described above wherein the reaction zone (12) comprises three pastilles, two serving as test pastilles and the last serving as a negative control pastille or a positive control pastille.

By “test pastille” is meant a biologically compatible pastille on which is lyophilized in situ all the reagents allowing the amplification and detection of at least one nucleic acid sequence of interest likely to be present in the biological sample to be tested. In this way, if a positive signal is obtained at the end of the test, then the sample contains the said at least one nucleic acid sequence of interest and the diagnosis is positive (subject to the results obtained on the control tablet(s)). In other words, in the current health context (i.e. COVID-19 pandemic), if a positive signal is obtained on this test pastille, the patient has an SARS-CoV-2 infection and the appropriate measures (medical treatment, confinement, etc.) can be taken. Furthermore, and assuming a diagnostic device (1) according to the invention comprising 3 tablets, 2 of which are test tablets, each test tablet can:
be prepared in such a way as to allow the amplification and detection of different nucleic acid sequences of interest of the same target (e.g. 2 test pastilles targeting 2 different regions of the same pathogen);
be prepared in such a way as to allow the amplification and detection of different nucleic acid sequences of interest from different targets (e.g. 2 test pastilles each targeting a specific region of two different pathogens).

Negative control pastille” means a biologically compatible pastille on which none of the reagents allowing the amplification and detection of at least one nucleic acid sequence of interest likely to be present in the biological sample to be tested is lyophilized. It also means a biologically compatible pastille on which one of the elements essential for the amplification or detection of at least one nucleic acid sequence of interest likely to be present in the biological sample to be tested is not lyophilized. Thus, this pastille at the end of the test may not positively reveal the presence of at least one nucleic acid sequence of interest. If this happens, the test is invalidated and must be repeated.

Positive control pastille” means a biologically compatible pastille on which is lyophilized in situ:
all the reagents allowing the amplification and detection of at least one nucleic acid sequence of interest likely to be present in the biological sample to be tested or of another nucleic acid sequence; and
said at least one nucleic acid sequence of interest or said other nucleic acid sequence.

In this way, this plate at the end of the test should show positively the presence of a nucleic acid sequence. If this does not happen, the test is invalidated and must be repeated. Advantageously, it should be noted that the nucleic acid sequence revealed by means of the positive control pastille and the lyophilized in situ reagents can be dissociated (different) from the said at least one nucleic acid sequence of interest likely to be present in the biological sample to be tested.

By “positive control pastille” is also meant, alternatively, a biologically compatible pastille on which is lyophilized in situ all the reagents allowing the amplification and detection of at least one other nucleic acid sequence of interest which is known to be necessarily present in the biological sample to be tested. In this way, this pastille at the end of the test should positively reveal the presence of said other nucleic acid sequence of interest. If this does not occur, the test is invalidated and must be repeated. For example, if the biological sample to be tested is saliva of human origin, the positive control pastille may be lyophilized in situ with all the reagents allowing the amplification and detection of at least one fragment of the gene coding for the human major histocompatibility complex, i.e. a fragment of the HLA gene. Also, if the biological sample to be tested is of human origin, “at least one other nucleic acid sequence of interest known to be necessarily present in the biological sample to be tested” means, for example, a fragment of a human ubiquitous housekeeping gene (i.e. transcribed and present in significant amounts in all cells) and exhibiting little interindividual variability. Common examples of such housekeeping genes are: human -actin, human Rnase-P, human ribosomal RNA (18S for example).

Among these embodiments, the invention thus relates to the diagnostic device (1) as described above in which the reaction zone (12) comprises:
a test pastille (13a or 13b) on which is lyophilized in situ all the reagents allowing the amplification and detection of at least one nucleic acid sequence of interest likely to be present in the biological sample to be tested; and
a negative control pastille (13b or 13a) on which:
   none of the reagents for the amplification and detection of at least one nucleic acid sequence of interest likely to be present in the biological sample to be tested is lyophilized; or
   one of the elements essential for the amplification or detection of at least one nucleic acid sequence of interest likely to be present in the biological sample to be tested is not lyophilized.

Among these embodiments, the invention also relates to the diagnostic device (1) as described above in which the reaction zone (12) comprises:
a test pastille (13a or 13b) on which is lyophilized in situ all the reagents allowing the amplification and detection of at least one nucleic acid sequence of interest likely to be present in the biological sample to be tested; and
a positive control pastille (13b or 13a) on which is lyophilized in situ:
   all the reagents allowing the amplification and detection of at least one nucleic acid sequence of interest likely to be present in the biological sample to be tested or of another nucleic acid sequence; and
   said at least one nucleic acid sequence of interest or said other nucleic acid sequence.

Among these embodiments, the invention also relates to the diagnostic device (1) as described above in which the reaction zone (12) comprises:
a test disc (13a or 13b) on which is lyophilized in situ all the reagents allowing the amplification and detection of at least one nucleic acid sequence of interest likely to be present in the biological sample to be tested; and
a positive control pastille (13b or 13a) on which is lyophilized in situ all the reagents allowing the amplification and detection of at least one other nucleic acid sequence of interest necessarily present in the biological sample to be tested.

Among these embodiments, the invention also relates to the diagnostic device (1) as described above in which the reaction zone (12) comprises:
a test pastille on which is lyophilized in situ all the reagents allowing the amplification and detection of at least one nucleic acid sequence of interest likely to be present in the biological sample to be tested;
a negative control pastille (13a or 13b) on which:
   none of the reagents for the amplification and detection of at least one nucleic acid sequence of interest likely to be present in the biological sample to be tested is lyophilized, or
   one of the elements essential for the amplification or detection of at least one nucleic acid sequence of interest likely to be present in the biological sample to be tested is not lyophilized; and
a positive control pastille (13b or 13a) on which is lyophilized in situ:
   all the reagents allowing the amplification and detection of at least one nucleic acid sequence of interest likely to be present in the biological sample to be tested or of another nucleic acid sequence; and
   said at least one nucleic acid sequence of interest or said other nucleic acid sequence.

Among these embodiments, the invention also relates to the diagnostic device (1) as described above in which the reaction zone (12) comprises:
a test pastille on which is lyophilized in situ all the reagents allowing the amplification and detection of at least one nucleic acid sequence of interest likely to be present in the biological sample to be tested;
a negative control pastille (13a or 13b) on which:
   none of the reagents for the amplification and detection of at least one nucleic acid sequence of interest likely to be present in the biological sample to be tested is lyophilized, or
   one of the elements essential for the amplification or detection of at least one nucleic acid sequence of interest likely to be present in the biological sample to be tested is not lyophilized; and
a positive control pastille (13b or 13a) on which is lyophilized in situ all the reagents allowing the amplification and detection of at least one other nucleic acid sequence of interest necessarily present in the biological sample to be tested.

Advantageously, the latter particular embodiments allow better reading of the results due to the color contrasts that can be observed between the three tablets. This also makes the interpretation of the results more secure. Alternatively, this embodiment comprising three pastilles may be described as the diagnostic device (1) as described above in which at least one preferably at least two bio-amplification pastilles (13a, 13b) contain freeze-dried reagents capable of allowing the amplification and detection of at least one nucleic acid sequence of interest likely to be present in said biological sample, and at least one of which also contains a freeze-dried nucleic acid of interest capable of being amplified by said freeze-dried reagents.

Alternatively, this embodiment comprising three pastilles may also be described as the diagnostic device (1) as described above wherein said at least one reaction zone (12) comprises at least two biologically compatible, adjacent and independent amplification pastilles (13a, 13b),
the first (13a or 13b) containing lyophilized reagents capable of allowing the amplification and detection of at least one nucleic acid sequence of interest likely to be present in said biological sample and
the second (13b or 13a) containing lyophilized reagents capable of allowing the amplification and detection of at least one other nucleic acid sequence of interest necessarily present in the biological sample to be tested.

As mentioned previously, “freeze-dried reagents capable of allowing the amplification and detection of at least one nucleic acid sequence of interest likely to be present in the above-mentioned biological sample” means molecular tools (primers possibly modified at the 5′ and/or 3′ ends) capable of hybridizing to a nucleic acid sequence of interest (DNA and/or RNA), amplifying the latter (enzymes, dNTPs) and visually (fluorescence, colorimetry, etc.) revealing its presence. Among these means are, but are not limited to, molecular tools allowing the implementation of isothermal nucleic acid amplification technologies (DNA and/or RNA) (e.g. RCA, LAMP, RPA, LAMP-QUASR, etc.) that do not require thermocycling. It should be noted that the amplification of RNA requires an upstream step of retro-transcription (RT) of the RNA into DNA by means of particular enzymes (i.e. viral retro-transcriptases, etc.), which step is also carried out at the level of the pastilles (13a and 13b) and for which the molecular tools can also be freeze-dried. In the sense of the invention, “amplification” is therefore understood to mean the step of retro-transcription of a target RNA (i.e. carrying said at least one nucleic acid sequence of interest) into DNA and the step of amplification of this DNA by a polymerase. Consequently, if the said at least one nucleic acid sequence of interest likely to be present in the sample to be tested comes from an RNA sequence (e.g. viral genomic RNA, the presence of which is to be detected), it is essential that the device include the means allowing its retro-transcription and amplification, and then its detection (e.g. RT-LAMP, RT-LAMP-QUASR, etc.).

According to another embodiment, the invention therefore concerns the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in the abovementioned biological sample, the said freeze-dried reagents being at least:
a polymerase;
dNTPs;
a set of at least six primers comprising primer F3, primer B3, primer FIP, primer BIP, primer LoopF and primer LoopB; and
an intercalating agent.

According to another embodiment, the invention relates to the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in said biological sample, said freeze-dried reagents being at least:
a retro-transcriptase;
a polymerase;
dNTPs;
a set of at least six primers comprising primer F3, primer B3, primer FIP, primer BIP, primer LoopF and primer LoopB; and
an intercalating agent.

According to another embodiment, the invention also concerns the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in the abovementioned biological sample, the said freeze-dried reagents being at least:
a polymerase;
dNTPs;
a set of at least six primers comprising primer F3, primer B3, primer FIP, primer BIP, primer LoopF and primer LoopB, at least one of which, with the exception of primer F3 and primer B3, is modified in the 5′ direction by the addition of a fluorophore; and
at least one oligonucleotide complementary to the FIP primer, BIP primer, LoopF primer or LoopB primer, said oligonucleotide being capable of reversibly coupling to the primer of which it is the complementary and said oligonucleotide being modified at the 3′ by a quencher.

According to a particular embodiment, the invention relates to the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in the abovementioned biological sample, the said freeze-dried reagents being at least:
a polymerase;
dNTPs;
a set of at least six primers comprising primer F3, primer B3, primer FIP, primer BIP, primer LoopF and primer LoopB, in which the primers FIP, BIP, LoopF and LoopB are modified in the 5′ direction by the addition of a fluorophore; and
four oligonucleotides complementary to the primers FIP, BIP, LoopF and LoopB, said oligonucleotides being capable of reversibly coupling to the primers to which they are complementary and said oligonucleotides being modified in the 3′ direction by a quencher.

According to another embodiment, the invention relates to the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in said biological sample, said freeze-dried reagents being at least:
a retro-transcriptase;
a polymerase;
dNTPs;
a set of at least six primers comprising primer F3, primer B3, primer FIP, primer BIP, primer LoopF and primer LoopB, at least one of which, with the exception of primer F3 and primer B3, is modified in the 5′ direction by the addition of a fluorophore; and
at least one oligonucleotide complementary to the FIP primer, BIP primer, LoopF primer or LoopB primer, said oligonucleotide being capable of reversibly coupling to the primer of which it is the complementary and said oligonucleotide being modified at the 3′ by a quencher.

According to a particular embodiment, the invention relates to the diagnostic device (1) as described above comprising at least one reaction zone (12) comprising at least one, preferably at least two, biologically compatible, adjacent and independent amplification pastilles (13a, 13b), at least one of which contains freeze-dried reagents capable of permitting the amplification and detection of at least one nucleic acid sequence of interest likely to be present in the abovementioned biological sample, the said freeze-dried reagents being at least:
a retro-transcriptase;
a polymerase;
dNTPs;
a set of at least six primers comprising primer F3, primer B3, primer FIP, primer BIP, primer LoopF and primer LoopB, in which the primers FIP, BIP, LoopF and LoopB are modified in the 5′ direction by the addition of a fluorophore; and
four oligonucleotides complementary to the primers FIP, BIP, LoopF and LoopB, said oligonucleotides being capable of reversibly coupling to the primers to which they are complementary and said oligonucleotides being modified in the 3′ direction by a quencher.

Among the freeze-dried reagents, MgSO₄, Betaine and Trehalose can be added and freeze-dried.

Retrotranscriptase, also known as reverse transcriptase, is an enzyme capable of converting RNA into DNA. This can be AMV retrotranscriptase or any other reverse transcriptase purified from a retrovirus or retrotransposon, or any other reverse transcriptase that has undergone directed evolutionary or other optimisation to improve its performance. In particular, the invention relates to the diagnostic device (1) as described above wherein said retrotranscriptase is AMV retrotranscriptase.

By “Polymerase” it is meant an enzyme capable of replicating DNA into DNA. This can be the Bst polymerase enzyme, the GspSSD polymerase enzyme or any other polymerase enzyme used in nucleic acid amplification (PCR or isothermal amplification type). In particular, the invention relates to the diagnostic device (1) as described above wherein said polymerase is selected from: the polymerase enzyme Bst and the polymerase enzyme GspSSD.

By “dNTPs” is meant the mixture of the four deoxyribonucleotides: dATP (deoxy adenine tri-phosphate), dCTP (deoxy cytosine tri-phosphate), dGTP (deoxy guanine tri-phosphate) and dTTP (deoxy thymine tri-phosphate).

By “a set of at least six primers” is meant nucleic acid sequences allowing isothermal amplification of at least one nucleic acid sequence of interest likely to be present in the biological sample to be tested. Furthermore, by “of which at least one ... is modified in the 5′ by the addition of a fluorophore”, it is meant that one, two, three or four primers are modified in the 5′ by the addition of a fluorophore, with the exception of primers F3 and B3 which are never modified. These primers modified in the 5′ by the addition of a fluorophore can also be called probes since they allow the detection of the signal. Preferably, the invention uses four of them, modified in the 5′, namely: the FIP primer, the BIP primer, the LoopF primer and the LoopB primer. In particular, these at least six primers can be:
primers of SEQ ID NOs: 1 to 6 allowing the detection of a nucleic acid sequence of interest of Dengue virus serotype 2 (DENV2); or
primers of SEQ ID NOs: 7 to 12 allowing the detection of a nucleic acid sequence of interest of the SARS-CoV-2 virus responsible for COVID-19 (Lin Yu et al., Rapid colorimetric detection of COVID-19 coronavirus using a reverse transcriptional loop-mediated isothermal amplification (RT-LAMP) diagnostic plat-form: iLACO; DOI:10.1101/2020.02.20.20025874).

By “Oligonucleotide” is meant a sequence of about 10 nucleotides. For example, an oligonucleotide of 5 to 20 nucleotides, i.e. 5, 6, 7, 8, 9, 10, 11, 12, 13, 14, 15, 16, 17, 18, 19 or 20 nucleotides. Complementary oligonucleotide” means an oligonucleotide that can specifically bind to a nucleic acid sequence. In the present case, these are oligonucleotides complementary to the primers FIP, BIP, LoopF and LoopB, of which at least one, or two, or three and preferably all four are modified in the 3′ direction by a quencher. The latter is a quencher capable of quenching the 5′-grafted fluorophore of a primer or primers FIB, BIP, LoopF and/or LoopB. In particular, this at least one and preferably four oligonucleotides are selected from the sequences SEQ ID NOs: 13 to 17 of which:
the sequence SEQ ID NO: 13 is complementary to the BIP primer of sequence SEQ ID NO: 4 used in the diagnosis of Dengue virus serotype 2 infection; and
SEQ ID NOs: 14 to 17 are respectively complementary to SEQ ID NOs: 9 to 12 used in the diagnosis of SARS-CoV-2 infection.

By “Intercalating agent” is meant a molecule capable of reversibly intercalating into the DNA and fluorescing when in the double helix. This can be SYBRGreen, a SYTO-type fluorophore (SYTO9, SYTO82, etc.), EvaGreen, ethidium bromide or any other DNA intercalating agent used in real-time nucleic acid amplification monitoring. In particular, the invention relates to the diagnostic device (1) as described above wherein said intercalating agent is selected from: SYBRGreen, a SYTO-type fluorophore (SYTO9, SYTO82, etc.), EvaGreen and ethidium bromide.

By “fluorophore”, also called fluorochrome, is meant a chemical or protein substance capable of emitting fluorescence light after excitation. In particular, the invention relates to the diagnostic device (1) as described above in which said fluorophore is selected from: fluorophores of the Alexa Fluor type (Alexa 350, Alexa 488, alexa 555, Alexa 647, etc. ), Cyanine type fluorophores (CY3, CY3.5, CY5, etc.), FAM type fluorophores (fluorescein amidite), Fluorescein Isothiocyanate (FITC), HEX type fluorophores, Texas Red type fluorophores and ATTO type fluorophores Preferably, FAM-type fluorophores and Texas Red fluorophores are used.

By “quencher”, also called deactivator, is meant a chemical or protein substance capable of deactivating (quenching) the excited state of a fluorophore. In particular, the invention relates to the diagnostic device (1) as described above wherein said quencher is selected from: DabCyl, BHQ-1, BHQ-2, BHQ-3, Cy5Q, Cy7Q, Iowa Black FQ, Iowa Black RQ, IRDye QC-1, QSY35, QSY7, QXL520, QXL570, QXL610 and QXL680. Preferably, the Iowa Black FQ and Iowa Black RQ quenchers are used.

It should be noted, however, that when using isothermal amplification technology and detection via a fluorophore/quencher system (e.g. (RT)-LAMP-QUASR), it is essential to choose the right pair. Table 1 below (Peng, X. et al., 2009, A nonfluorescent, broad-range quencher dye for Förster resonance energy transfer assays. Analytical biochemistry, 388(2), 220-228) is used to make this choice. For example, if the fluorophore used is Texas Red then it is preferable to use Iowa Black RQ.

[image] As described above, the diagnostic device (1) of the invention comprises a single detection unit (4) and allows only one biological sample to be tested. Advantageously, it can be configured to accommodate different detection units distributed over the upper (3) and lower (5) discs. This configuration thus offers the user a very practical multi-sample device due to its versatility since, depending on its configuration, it can be used:
or from a sample, to carry out different diagnoses (e.g. Zika virus, SARS-CoV-2, HIV, Salmonella, etc.);
or from several samples, to carry out a single type of diagnosis (e.g. Zika virus or SARS-CoV2 or HIV or Salmonella, etc.);
or from several samples, to perform different diagnoses (e.g. Zika virus, SARS-CoV-2, HIV, Salmonella, etc.).

According to another embodiment, the invention thus relates to the diagnostic device (1) as described above comprising at least two detection units which are distributed over the upper (3) and lower (5) discs in such a way that their respective receiving openings are located on the same radius of the upper disc.

According to another embodiment, the invention thus relates to the diagnostic device (1) as described above comprising at least two detection units which are distributed on the upper (3) and lower (5) discs in such a way that their respective receiving openings are located on the same arc of a circle of the upper disc.

According to another embodiment, the invention thus relates to the diagnostic device (1) as described above comprising:
at least two detection units which are distributed over the upper (3) and lower (5) discs in such a way that their respective receiving openings lie on the same radius of the upper disc, and
at least two detection units which are distributed over the upper (3) and lower (5) discs in such a way that their respective receiving openings are located on the same arc of the upper disc.

According to another embodiment, the invention thus relates to the diagnostic device (1) as described above comprising:
at least two detection units which are distributed over the upper (3) and lower (5) discs in such a way that their respective receiving openings lie on the same radius of the upper disc, or
at least two detection units which are distributed over the upper (3) and lower (5) discs in such a way that their respective receiving openings are located on the same arc of the upper disc.

Advantageously and according to this multi-sample configuration, the windows (9) arranged on the upper disc (3) of the detection units arranged on the same radius can be grouped together and form only one. The same applies to any recesses (6) arranged on the same radius on the lower disc (5), which can be combined to form one recess. According to another embodiment, the invention thus relates to the diagnostic device (1) as described above in which:
the windows (9) arranged on the same radius of the upper disc (3) are grouped together and form a single window, and/or
any recesses (6) arranged on the same radius of the lower disc (5) are grouped together and form a single disc.

Alternatively, and with regard to this multi-sample configuration, the invention can be defined as a portable diagnostic device (1) in the form of a flat closed cylindrical housing comprising:
a lower disc (5); and
an upper disc (3),
the two discs being:
mounted on each other and movable relative to each other so that the upper disc (3) is capable of rotating on the lower disc (5) in both possible directions of rotation; and
segmented into x diagnostic blocks (2), where x is an integer greater than or equal to 1 and each diagnostic block comprises y detection units (4) of samples to be tested, each detection unit comprising:
   a receptacle comprising an absorbent material (15) disposed either on the upper surface of the lower disc (5) or integrated into the mass of said upper surface of the lower disc (5), and said absorbent material (15) being capable of holding a volume of at least 1 mL;
   a reaction zone (12) disposed in the lower disc, said reaction zone (12) comprising at least one preferably at least two biologically compatible, adjacent and independent pastilles (13a and 13b) on a same circle of the lower disc (5) or a same radius of the lower disc (5), and of which at least one optionally comprises on its surface freeze-dried reagents allowing the amplification and detection of at least one nucleic acid sequence of interest likely to be present in a sample to be tested;
   possibly a recess (6) arranged in the lower disc (5), said recess (6) being provided with a sealing material (10), said recess (6) provided with a sealing material (10) being in line with the reaction zone (12) and integral therewith, and in particular allowing light to reach said at least one preferably at least two biologically compatible pastilles (13a and 13b), and said sealing material (10) being either removably positioned on the recess (6) or integrated into the mass of the lower disc (5);
   a receiving opening (7) disposed in the upper disc (3), said opening being:
      positioned on a circle of the upper disc, which circle is parallel to the circle of the lower disc (3) on which said reaction zone (12) is positioned;
      equipped with a biologically compatible membrane (8) allowing the extraction and retention of nucleic acids from the test sample in the presence of appropriate reagents; and
      capable of being positioned in line with the above reaction zone and of covering it in part or in full; and
   a window (9) provided in the upper disc (3), said window (9) being provided with an optical filter (11), said window (9) provided with an optical filter (11) being able to be positioned in line with the reaction zone (12) of the detection unit (4), and said optical filter (11) being either removably positioned on the window (9) or integrated into the mass of the upper disc (3),
where y is an integer greater than or equal to 1, and when y is greater than or equal to 2, the individual elements of the lower disc (5) and the upper disc (3) of the detection units (4) are respectively adjacent and independent along a respective radius of the lower disc (5) and the upper disc (3), and each diagnostic block (2) being configured so that the upper disc (3) can, by rotation through an angle of at least 30°, pass:
a first position in which:
   the receiving opening (7) equipped with a biologically compatible membrane (8) of a detection unit (4) is located adjacent to the absorbent material (15) of said detection unit (4) to allow extraction and retention of nucleic acids from said test sample; and
   the window (9) provided with an optical filter (11) of said detection unit (4) is located at the reaction area (12) of said detection unit (4),
to a second position in which:
   the receiving opening (7) equipped with a biologically compatible membrane (8) of said detection unit (4) is located in front of the reaction zone (12) of said detection unit (4) and covers it in part or in whole to allow the elution of nucleic acids from said test sample in the presence of the appropriate reagents, said nucleic acids by gravity ending up on said at least one preferably at least two biologically compatible pastilles (13a and 13b) of said detection unit (4); and
   the window (9) provided with an optical filter (11) of said detection unit (4) is offset by an angle of at least 30°,
then to position itself in said first position so that the window (9) provided with an optical filter (11) of said detection unit (4) is again in line with the reaction zone (12) of said detection unit (4) to form a substantially hermetic and substantially sealed amplification and detection chamber, said chamber allowing the amplification and detection of at least one nucleic acid sequence of interest likely to be present in the sample to be tested.

In the same way, the passage from the above-mentioned second position to the above-mentioned first position can thus be made either by a rotation opposite to that allowing the passage from the above-mentioned first position to the above-mentioned second position, or by a rotation in the same direction as the latter. Advantageously, the invention relates to the portable diagnostic device (1) in the form of a flat closed cylindrical housing as described above comprising:
a lower disc (5); and
an upper disc (3),
the two discs being:
mounted on each other and movable relative to each other so that the upper disc (3) is capable of rotating on the lower disc (5) in both possible directions of rotation; and
segmented into x diagnostic blocks (2), where x is an integer greater than or equal to 1 and each diagnostic block comprises v detection units (4) of samples to be tested, each detection unit comprising:
   a receptacle comprising an absorbent material (15) disposed either on the upper surface of the lower disc (5) or integrated into the mass of said upper surface of the lower disc (5), and said absorbent material (15) being capable of holding a volume of at least 1 mL;
   a reaction zone (12) disposed in the lower disc, said reaction zone (12) comprising at least one preferably at least two biologically compatible, adjacent and independent pastilles (13a and 13b) on a same circle of the lower disc (5) or a same radius of the lower disc (5), and of which at least one optionally comprises on its surface freeze-dried reagents allowing the amplification and detection of at least one nucleic acid sequence of interest likely to be present in a sample to be tested;
   possibly a recess (6) arranged in the lower disc (5), said recess (6) being provided with a sealing material (10), said recess (6) provided with a sealing material (10) being in line with the reaction zone (12) and integral therewith, and in particular allowing light to reach said at least one preferably at least two biologically compatible pastilles (13a and 13b), and said sealing material (10) being either removably positioned on the recess (6) or integrated into the mass of the lower disc (5);
   a receiving opening (7) disposed in the upper disc (3), said opening being:
      positioned on a circle of the upper disc, which circle is parallel to the circle of the lower disc (3) on which said reaction zone (12) is positioned;
      equipped with a biologically compatible membrane (8) allowing the extraction and retention of nucleic acids from the test sample in the presence of appropriate reagents; and
      capable of being positioned in line with the above reaction zone and of covering it in part or in full; and
   a window (9) provided in the upper disc (3), said window (9) being provided with an optical filter (11), said window (9) provided with an optical filter (11) being able to be positioned in line with the reaction zone (12) of the detection unit (4), and said optical filter (11) being either removably positioned on the window (9) or integrated into the mass of the upper disc (3),
where y is an integer greater than or equal to 1, and when y is greater than or equal to 2, the individual elements of the lower disc (5) and the upper disc (3) of the detection units (4) are respectively adjacent and independent along a respective radius of the lower disc (5) and the upper disc (3), and each diagnostic block (2) being configured so that the upper disc (3) can, by rotation through an angle of at least 30°, pass:
a first position in which:
   the biologically compatible membrane-equipped receiving opening (7) of a detection unit (4) is in register with the absorbent material (15) of said detection unit (4) to allow extraction and retention of nucleic acids from said test sample; and
   the window (9) provided with an optical filter (11) of said detection unit (4) is located at the reaction area (12) of said detection unit (4),
to a second position in which:
   the receiving opening (7) equipped with a biologically compatible membrane (8) of said detection unit (4) is located in front of the reaction zone (12) of said detection unit (4) and covers it in part or in whole to allow the elution of nucleic acids from said test sample in the presence of the appropriate reagents, said nucleic acids by gravity ending up on said at least one preferably at least two biologically compatible pastilles (13a and 13b) of said detection unit (4); and
   the window (9) provided with an optical filter (11) of said detection unit (4) is offset by an angle of at least 30°,
then to return by reverse rotation to the said first position so that the window (9) provided with an optical filter (11) of the said detection unit (4) is again in line with the reaction zone (12) of the said detection unit (4) to form a substantially hermetic and substantially sealed amplification and detection chamber, the said chamber allowing the amplification and detection of at least one nucleic acid sequence of interest likely to be present in the sample to be tested,
and each diagnostic block (2) also being configured such that the window or windows (9a and 9b) provided with an optical filter (11) of a detector unit or units (4) of a diagnostic block (2) cannot be in line with the receptacle or receptacles comprising absorbent material (15) of said diagnostic block (2) or another diagnostic block (2′) irrespective of the position of the upper disc (3) relative to the lower disc (5).

As all embodiments are compatible with each other and can be combined with each other, they apply to this alternative definition of the invention. The reverse is also true. Furthermore, all the definitions provided apply to all the embodiments described.

By “x being an integer greater than or equal to 1”, it is meant that x can be equal to 1, 2, 3, 4, 5, 6, 7, 8, 9 or 10. It is also understood that x can be from 1 to 10, from 1 to 5, from 2 to 10, from 4 to 8. The term “y being an integer greater than or equal to 1” means that y may be 1, 2, 3, 4, 5, 6, 7, 8, 9, 10, 11, 12, 13, 14, 15, 16, 17, 18, 19 or 20. It is also understood that y can be 1 to 20, 1 to 6, 1 to 12, 1 to 5, 1 to 10, 5 to 15 or 10 to 20.

By “An angle of at least 30°” it is meant that the rotation of the upper disc (3) on the lower disc (5) may be 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, 180°, 185°, 195°, 200°, 205°, 210°, 215°, 220°, 225°, 230°, 235°, 240°, 245°, 250°, 255°, 260°, 265°, 270°, 275°, 280°, 285°, 295°, 300°, 305°, 310°, 315° or 320° or even 360° allowing the upper disc (3) to rotate freely (i.e. make a complete turn) on the upper disc.(i.e. make a complete turn) on the lower disc (5) and vice versa. An angle between 30° and 320° is also meant. Of course, it should be noted that the possible angle of rotation is dependent on the number of diagnostic blocks (2) which are arranged on the diagnostic device (1) of the invention, so that all the diagnostic blocks preferably operate simultaneously.

In particular, according to this alternative the invention relates to the diagnostic device (1) as described above in which x and y are selected from the pairs:
x = 1 and y = 1 (corresponding to the configuration of a biological sample);
x = 1 and y is from 1 to 6;
x = 2 and y is from 1 to 6;
x = 3 and y is from 1 to 6.

According to this alternative, the invention advantageously relates to the diagnostic device (1) as described above in which each of the x diagnostic blocks (2) comprises y detection units (4) of which all the y windows (9) provided with an optical filter (11) arranged on the upper disc (3) form one.

According to this alternative, the invention advantageously relates to the diagnostic device (1) as described above in which each of the x diagnostic blocks (2) comprises y detection units (4) of which all of the y possible recesses (6) provided with a sealing material (10) arranged on the lower disc (5) form one.

According to this alternative, the invention also relates to the diagnostic device (1) as described above in which each of the x diagnostic blocks (2) comprises y detection units (4) of which the set of y windows (9) provided with an optical filter (11) arranged on the upper disc (3) forms one and

of which the whole of the possible ones (6) provided with a sealing material (10) arranged on the lower disc (5) form one.

According to another embodiment, the invention relates to the diagnostic device (1) as described above further comprising means, in particular:
at least one clamp capable of gripping said device and applying pressure to lock and clamp the upper disc to the lower disc, said pressure ensuring hermeticity and sealing of the device, and equipped with a heating system capable of being positioned in line with the reaction zone(s) of the device avoiding the creation of a temperature gradient on the reaction zone(s) to be heated.

A second aspect of the invention concerns the use of the portable diagnostic device (1) in the form of a cylindrical housing as described above to detect in vitro at least one nucleic acid sequence of interest which may be present in a biological sample to be tested. This at least one nucleic acid sequence (DNA and/or RNA) of interest may be:
a nucleic acid sequence present in the genome of a pathogen (virus, bacterium, microorganism, etc.), or
a nucleic acid sequence present in the genome (DNA) or transcriptome (RNA) of a mammal (e.g. human), the presence of which is linked to a predisposition to a disease,
the use according to the invention allows the diagnosis of infections due to one or more pathogens and also the diagnosis of genetic diseases. This usefulness and versatility of the diagnostic device (1) of the invention are of course conditioned by the reagents (molecular tools) possibly lyophilized on the surface of one or more so-called “test” pastilles. For example, if all the primers allowing the detection of at least one nucleic acid sequence of the SARS-CoV-2 coronavirus are freeze-dried (or added extemporaneously), the use described makes it possible to diagnose an infection with this virus. Similarly, if it is freeze-dried (or added extemporaneously), the set of primers allowing the detection of at least one nucleic acid sequence of the Dengue virus serotype 1, the use described allows the diagnosis of an infection with this virus. Etc.

Advantageously, the multi-sample configuration allows one or more biological samples to be tested for several types of infection. For example, in the configuration where x = 2 and y = 3, a diagnostic block would be used to test a first biological sample in vitro for influenza virus, COVID-19 virus (SARS-CoV-2) and/or Staphylococcus aureus infection, and

the second diagnostic block would be used to test a second biological sample in vitro for influenza virus, COVID-19 (SARS-CoV-2) and/or Staphylococcus aureus infection. This example can be generalized to x diagnostic blocks for x biological samples, each of the x blocks comprising y detection units for testing in vitro for the presence (or not) of y viral, bacterial or other microorganism (e.g. fungal) infections. Similarly, the diagnostic device (1) of the invention can be used to test in vitro y genetic diseases.

According to another embodiment, the invention thus relates to the use as described above for detecting in vitro a viral infection (e.g. HIV, Dengue virus, Zika virus, Sigma 3 virus, SARS-CoV-2, etc.), a bacterial infection (e.g. Salmonella, Pseudomonas, Staphylococcus, Escherichia, Streptococcus, Helicobacter, etc.), a microorganism-related infection (e.g. fungus) and/or a genetic disease (e.g. cystic fibrosis, neurofibromatosis type 1, haemophilia, trisomyelitis, etc.). ), a microorganism-related infection (e.g. fungus) and/or a genetic disease (e.g. cystic fibrosis, neurofibromatosis type 1, haemophilia, trisomy 21, myopathy, etc. ). In particular, the invention relates to the use as described above for detecting a viral infection in vitro. The invention also relates to the use as described above for detecting in vitro a viral infection and in particular for detecting in vitro a SARS-CoV-2 viral infection. Preferably, the invention relates to the use as described above for detecting in vitro SARS-Cov-2 viral infection. As mentioned above, this embodiment of the invention is conditioned by the use of, for example, the primers of sequences SEQ ID NO: 7 to 12 on the test pastille present on the reaction zone (12).

Another aspect of the invention concerns the method of in vitro detection of at least one nucleic acid sequence of interest likely to be present in a biological sample to be tested, said method being implemented with the aid of the portable diagnostic device (1) in the form of a cylindrical housing as described above.

In particular, the invention relates to the method of in vitro detection of at least one nucleic acid sequence of interest likely to be present in a biological sample to be tested, said method being implemented with the aid of the portable diagnostic device (1) in the form of a cylindrical housing as described above and said method comprising at least the following steps:
a. extracting nucleic acids from the biological sample to be tested on a biologically compatible membrane (8) of a detection unit (4);
b. turning the upper disc (3) to position said biologically compatible membrane (8) over the reaction zone (12) of that detection unit and cover it in part or in whole;
c. eluting nucleic acids from said biologically compatible membrane (8) to the at least one preferably at least two biologically compatible pastilles (13a, 13b) of said detection unit (4);
d. turning the upper disc (3) to return to the initial position; and
e. amplifying and then detect whether amplification of at least one nucleic acid sequence of interest that may be present in a biological sample has occurred.

According to the configuration of the diagnostic device (1) of the invention, the above-mentioned step d. may be carried out either by a rotation in the opposite direction to that of step b., or by a rotation in the same direction as that of step b. Advantageously, the invention relates to the method of in vitro detection of at least one nucleic acid sequence of interest likely to be present in a biological sample to be tested, said method being implemented with the aid of the portable diagnostic device (1) in the form of a cylindrical housing as described above and said method comprising at least the following steps:
a. extracting nucleic acids from the biological sample to be tested on a biologically compatible membrane (8) of a detection unit (4);
b. turning the upper disc (3) to position said biologically compatible membrane (8) over the reaction zone (12) of that detection unit and cover it in part or in whole;
c. eluting nucleic acids from said biologically compatible membrane (8) to the at least one preferably at least two biologically compatible pastilles (13a, 13b) of said detection unit (4);
d. turning the upper disc (3) in the opposite direction to return to the initial position; and
e. amplifying and then detect whether amplification of at least one nucleic acid sequence of interest that may be present in a biological sample has occurred.

Since the above method involves the use of the diagnostic device (1) of the invention, everything that applies to the uses (definitions, etc.) also applies to the methods.

According to another embodiment, the invention relates to the process as described above, said process comprising at least the following steps:
a. depositing the biological sample to be tested on a biologically compatible membrane (8) of a detection unit (4);
b. extracting nucleic acids from the biological sample to be tested at this biologically compatible membrane (8);
c. washing said biologically compatible membrane (8) from said detection unit (4);
b. turning the upper disc (3) to position said biologically compatible membrane (8) over the reaction zone (12) of that detection unit and cover it in part or in whole;
c. drying said biologically compatible membrane (8) from said detection unit (4)
d. eluting nucleic acids from said biologically compatible membrane (8) to the at least one preferably at least two biologically compatible pastilles (13a, 13b) of said detection unit (4);
e. turning the upper disc (3) in the same direction as in step b. or in the opposite direction to step b. to return to the initial position;
f. heating the reaction zone (12) to 65° C. for 40 minutes to amplify the at least one nucleic acid sequence of interest likely to be present in the biological sample; and
g. detecting whether amplification of at least one nucleic acid sequence of interest that may be present in a biological sample has occurred.

Whether the invention relates to uses as described above or to methods as described above, the observation of a positive signal (e.g. fluorescent light emission, appearance of a stain) at the test pastille means that the at least one nucleic acid sequence (DNA and/or RNA) has been amplified and is present in the biological sample. For example, if it was intended to test in vitro for SARS-CoV-2 infection in a patient, the observation of this positive signal means that the patient is infected with SARS-CoV-2 coronavirus and is suffering from COVID-19. Conversely, for example, if it were desired to test a patient in vitro for SARS-CoV-2 infection, failure to observe a positive signal means that the patient is not infected with SARS-CoV-2 coronavirus and does not have COVID-19. However, to ensure the veracity of this in vitro test, the results of a positive control and/or a positive control, which can be performed outside the diagnostic device (1) of the invention, should also be considered. Preferably, these controls are integrated into the diagnostic device (1) of the invention via the positive control pastille which must always reveal a positive signal and/or via the negative control pastille which must always reveal a negative signal (i.e. absence of fluorescence, absence of color). If this is not the case at the end of the test, the test is invalidated and must be repeated. The presence of a second tablet on the reaction zone is therefore preferred for the reliability and safety of the in vitro diagnostic test that the diagnostic device (1) of the invention makes possible. Furthermore, it should be noted that the observation of the signals on each of the pastilles obtained by colorimetry, fluorometry, etc., can be carried out by means of the device (1). It should also be noted that the observation of the signals on each of the pastilles obtained by colorimetry, fluorometry, etc., can be carried out by means of the human eye, or by a camera or photographic apparatus conventionally used in a laboratory or hospital. It can even be a smartphone camera. In addition, and depending on the detection technology chosen, it is possible to carry out kinetic monitoring of the test (e.g. (RT)-LAMP) or “end point” monitoring (e.g. (RT)-LAMP-QUASR).

Another aspect of the invention relates to the method of manufacturing (e.g. industrially) the portable cylindrical housing-shaped diagnostic device (1) as described above. In particular, the invention relates to the method of manufacturing the portable diagnostic device (1) in the form of a cylindrical housing as described above, said method comprising at least the following steps:
a. manufacturing (e.g. 3D printing or injection moulding) the upper (3) and lower (5) discs;
b. arranging at least one biologically compatible membrane (8), at least one preferably at least two biologically compatible pastilles (13a and 13b), at least one absorbent material (15) and at least one optical filter (11); and
c. assembling the upper (3) and lower (5) discs.

According to another embodiment, the invention relates to the manufacturing process as described above, said process comprising at least the following steps:
a. manufacturing (e.g. 3D printing or injection moulding) the upper (3) and lower (5) discs;
b. disposing at least one biologically compatible membrane (8), at least one preferably at least two biologically compatible pastilles (13a and 13b), at least one absorbent material (15), at least one optical filter (11) and optionally at least one sealing material (10); and
c. assembling the upper (3) and lower (5) discs.

According to another embodiment, the invention relates to the manufacturing process as described above, said process comprising at least the following steps:
a. manufacturing (e.g. 3D printing) the upper (3) and lower (5) discs;
b. washing the upper (3) and lower (5) discs with RNA free solution;
c. arranging at least one biologically compatible membrane (8), at least one preferably at least two biologically compatible pastilles (13a and 13b), at least one absorbent material (15), at least one optical filter (11) and optionally at least one sealing material (10); and
d. assembling the upper (3) and lower (5) discs.

According to another embodiment, the invention relates to the manufacturing process as described above, said process comprising at least the following steps:
a. manufacturing (e.g. 3D printing) the upper (3) and lower (5) discs;
b. washing the upper (3) and lower (5) discs with RNA free solution;
c. arranging at least one biologically compatible membrane (8), at least one preferably at least two biologically compatible pastilles (13a and 13b), at least one absorbent material (15), at least one optical filter (11) and possibly at least one sealing material (10);
d. assembling the upper (3) and lower (5) discs; and
e. freeze-drying on at least one pastille (13a or 13b) reagents capable of allowing the amplification and detection of at least one nucleic acid sequence of interest likely to be present in said biological sample.

Alternatively, the invention relates to the manufacturing process as described above, said process comprising at least the following steps:
a. manufacturing (e.g. 3D printing) the upper (3) and lower (5) discs;
b. freeze-drying on at least one pastille (13a or 13b) reagents capable of allowing the amplification and detection of at least one nucleic acid sequence of interest likely to be present in said biological sample.
c. washing the upper (3) and lower (5) discs with RNA free solution;
d. arranging at least one biologically compatible membrane (8), at least one preferably at least two biologically compatible pastilles (13a and 13b), at least one absorbent material (15), at least one optical filter (11) and optionally at least one sealing material (10); and
e. assembling the upper (3) and lower (5) discs.

According to the needs of the user, variants of the invention can be developed, which take up and/or integrate and/or can be combined with the aspects (definitions) described above.

One of them concerns the automatic implementation of the diagnostic device of the invention. In particular, this can be implemented by using two devices, namely a first device, hereinafter referred to as “dispenser” (20), and a second device, hereinafter referred to as “detector” (21). For the purposes of the invention, these two devices may be used independently of each other, or in combination and will then be referred to hereinafter as the “diagnostic system” (22). It should be noted that the diagnostic device (1, 31), the dispenser (20) and the detector (21) can of course be used in combination or individually.

FIGS. 12 and 13 show an example of a dispenser (20) according to the invention. In this example it is in the form of a housing comprising a base (23) closed by a cover (24) hinged about a hinge. The base (23) is hollowed out with a circular cavity (25) intended to receive a diagnostic device (31), the bottom of this cavity (25) being provided with means, not shown on the drawing, ensuring the immobilization in rotation of the base of the diagnostic device (31), as explained below.

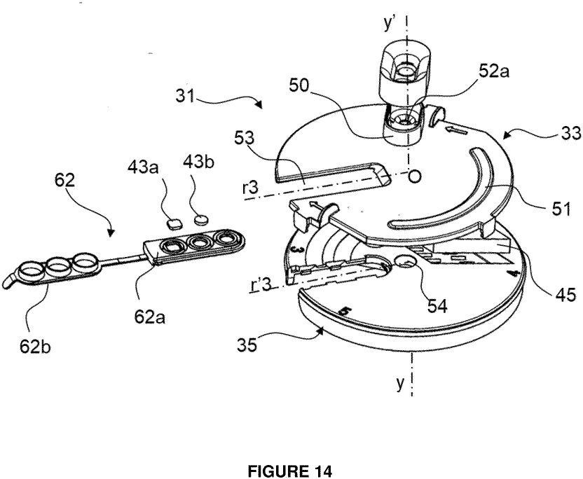

The diagnostic device (31), which is shown in an exploded view in FIG. 14, has some of the essential elements of the diagnostic device (1) described above with some adjustments to ensure its operation within automated means. For clarity of description, the same references are kept for similar elements, increased by a value of 30.

The diagnostic device (31) thus comprises two superimposed concentric discs, namely an upper disc (33) and a lower disc (35), which are mounted for rotation about an axis yy′ perpendicular to their centre O.

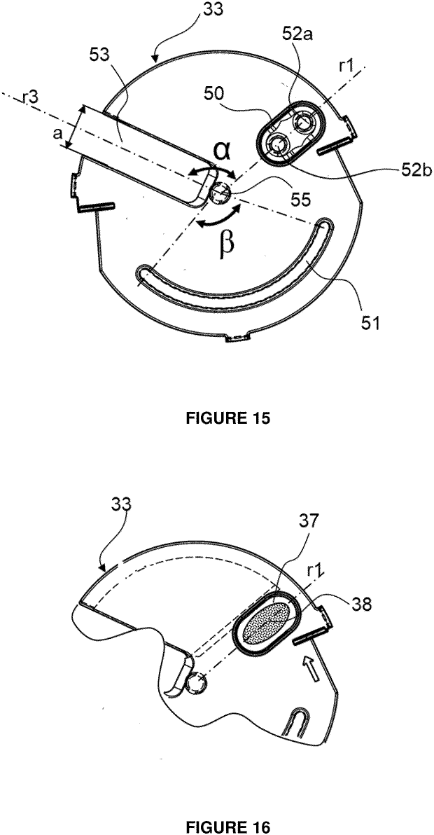

The upper disc (33) has a collection area formed by a cylindrical boss (50) whose base is substantially oval in shape. This boss is hollowed out by a recess whose base is pierced by two receiving openings (52a and 52b) which are each provided with a capture membrane (38), respectively (38a and 38b), which is biologically compatible and able to allow the extraction and retention of nucleic acids from the biological sample taken in the presence of appropriate reagents. These two receptor openings (52a and 52b) are arranged slightly apart from each other on the same radius r1 of the disc (33), as shown in FIG. 15.

Of course, as in the previous embodiment, a single opening (37) elongated along the radius r₁ and closed by a biologically compatible membrane (38) could be made, as shown in the partial FIG. 16.

On the cylindrical boss (50), a removable funnel (49) can be positioned, equipped with two openings which are arranged opposite the two openings (52a and 52b). This funnel can also be equipped with a single opening. This funnel makes it possible to avoid contamination. Indeed, the funnel (49) serves to prevent contact of the lysis product with the sample deposition area (the part covered by the funnel) and its use is preferred. If this is not the case in manual mode, then there is a risk that the lysate will wet the sample deposition area. When the rinsing agent (e.g. 70% ethanol) is then applied, it can mix with any traces of lysate remaining on the surface of the sample deposit area. The rinsing agent then becomes contaminated with lysate. After rinsing, traces of lysis product would remain on the membranes, which would be eluted into the reaction membranes with the RNA captured during the elution step. Finally, if this happens, an undesired inhibition of the amplification reaction would be created, which can be avoided by the use of the funnel (49), hence its interest.

In the case of automatic use, sample removal is currently no different from that in the case of manual use. The same constraints apply and the use of the funnel (49) is recommended for the reasons given above.

The upper disc (33) is pierced by a radial slot (53) of axis r₃ and width a, the function of which will be explained below, and which forms with the axis r₁ an angle at the centre α the value of which is at least equal to 30° and preferably in this example of the order of 60°. It is also pierced near its periphery by a circular slot (51) extending at an angle at the centre β slightly greater than the angle α, the function of which will be specified below.

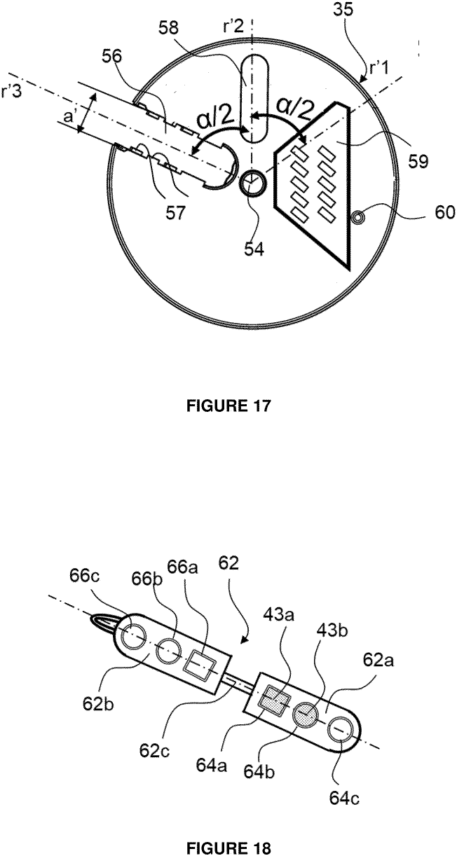

The lower disc (35), as shown in FIGS. 14 and 17, has a central hole (54) which is intended to receive a central pin (55) made under the upper disc (33), thus ensuring the relative rotation of the two discs. The lower disc (33) has a tenon (60) on its upper side which is arranged in such a way that it fits into the circular slot (51) of the upper disc (33) when the two discs are superimposed in the operating position. It is understood in these conditions that the value of the angle at the centre β of the circular slot (51) makes it possible to control the minimum and maximum angle of rotation of the upper disc (33) with respect to the lower disc (35).

As shown in FIGS. 14 and 17, the lower disc (35) is pierced by a radial slot (56) of axis r′3 and width a′ slightly less than the width a of the radial slot (53) of the upper disc. The sides of this slot are provided with gripping means, such as teeth (57), which are intended to hold a capsule, as explained below. The lower disc (35) is also traversed by a radial window (58) of axis r′2 which forms an angle at the centre with the axis r′3 of the slot (56) equal, in this example, to α/2.

The lower disc (35) is provided with a substantially trapezoidal shaped small tank (59) which is intended to receive an absorbent material (45) of a certain volume, so as to allow the extraction and retention of nucleic acids, and to collect the overflow of the biological sample to be tested and of the nucleic acid extraction and washing reagents. This small tank (59) is arranged such that a radial axis r′1 forming with the axis r′2 an angle at the centre equal to α/2 passes through said small tank (59), as shown in FIG. 17.

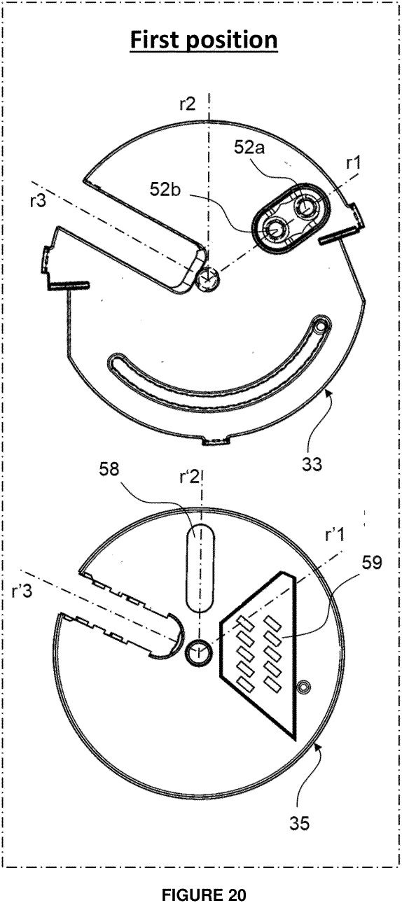

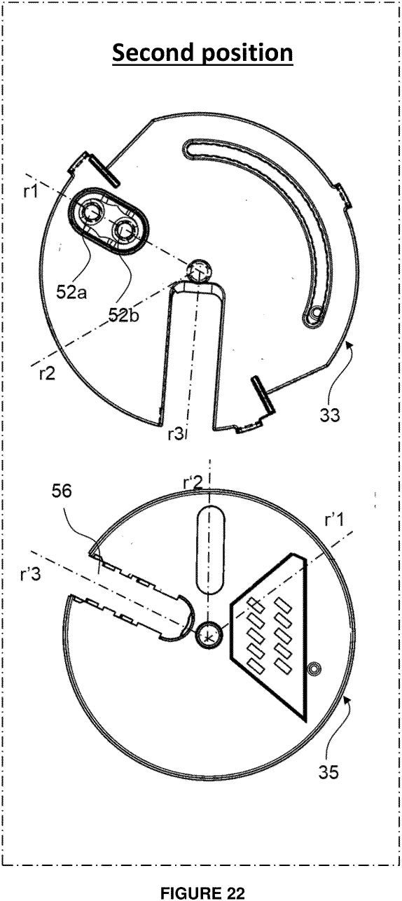

According to the invention, the angle at the centre β defining the dimensions of the circular slot (51) is given a value such that, when the axis r1 of the upper disc is in line with the axis r′1 of the lower disc, the tenon (60) is in abutment against a first end of the groove (51), thus preventing any clockwise rotation of the upper disc, as shown in FIG. 20, and that, when the axis r1 of the upper disc is at right angles to the axis r′3 of the lower disc, the tenon (60) is in abutment against the second end of the groove (51), as shown in FIG. 22, thus preventing any counter-clockwise rotation of the upper disc.

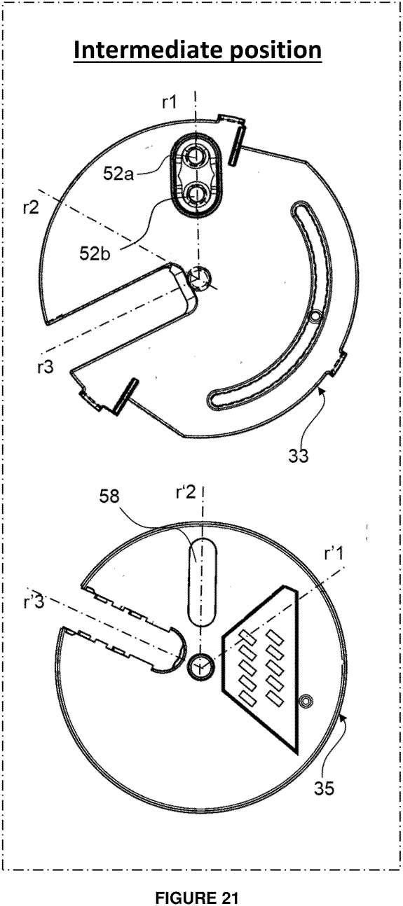

As described, it is thus understood that the upper disc (33) is able to occupy three remarkable positions with respect to the lower disc (35), namely:
a first position, shown in FIG. 20, in which the receiving openings (52a and 52b) are located in line with the small tank (59) intended to contain the absorbent material (45) (superposition of the axes r1 and r′1);
a second position shown in FIG. 22 in which the receiving openings (52a and 52b) are in line with the radial slot (56) in the lower disc (35) for receiving a capsule (62) (superposition of axes r1 and r′3); and
an intermediate position, shown in FIG. 21, in which the receiving openings (52a and 52b) are located in line with the radial window (58) (superposition of the axes r1 and r′2).

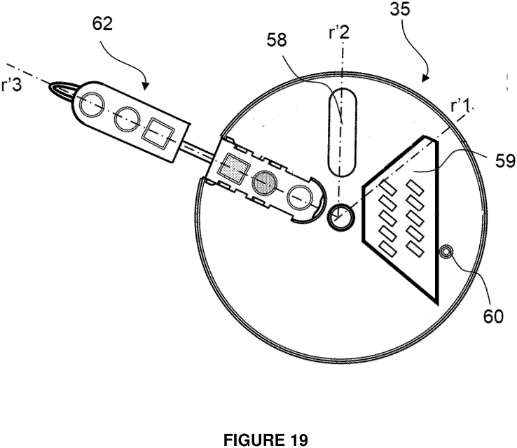

This capsule (62), which may be made of a material selected from: polypropylene and in particular food grade polypropylene, and which is shown in FIGS. 14 and 18; has an overall elongated configuration which allows it to fit into the radial slot (56) and to be held therein, as shown in FIG. 19. The inner part of the capsule has three hollow bosses, respectively (64a, 64b, and 64c). The two outermost hollow bosses contain reagent pastilles, respectively (43a and 43b), which are biologically compatible, independent, and capable of allowing the amplification and detection of at least one nucleic acid sequence of interest likely to be present in the biological sample in the presence of appropriate reagents.

The part (62a) of the capsule (62) suitable for insertion into the radial slot (56) is extended by a cover (62b) which is connected to it by a link (62c). The inner face of this cover also comprises hollow bosses (66a, 66b and 66c), the internal diameter of which is slightly greater than the external diameter of the bosses (64a, 64b and 64c) so that they can be fitted onto the latter in order to close them off.

Of course, the capsule (62) could have any other shape and contain more than two reagent tablets. Advantageously, the cover (62b) of the capsule (62) is made in particular of a transparent material, in particular a synthetic material which is dyed yellow.

As previously indicated and shown in FIG. 13, the cavity (25) of the dispenser (20) is adapted to receive the diagnostic device (31) and to ensure that the lower disc (35) is secured against rotation. The upper part of the cover (24) has a rotatable disc (68) with indentations to enable it to fit over the upper disc (33) and to engage the latter when the cover (24) is closed. The rotating disc (68) is rotated by a motor, in particular a stepping motor, which is driven by a control logic under the control of a microcontroller in particular, and which is able to put the upper disc (33) in the three remarkable positions mentioned above, namely:
the first position in which the receiving openings (52a and 52b) are located above the absorbent material (45) (superposition of the axes r1 and r′1);
the second position in which the receiving openings (52a and 52b) are located above the capsule (62) (superposition of axes r1 and r′3); and
the intermediate position in which the receiving openings (52a and 52b) are located above the radial window (58) (superposition of the axes r1 and r′2).

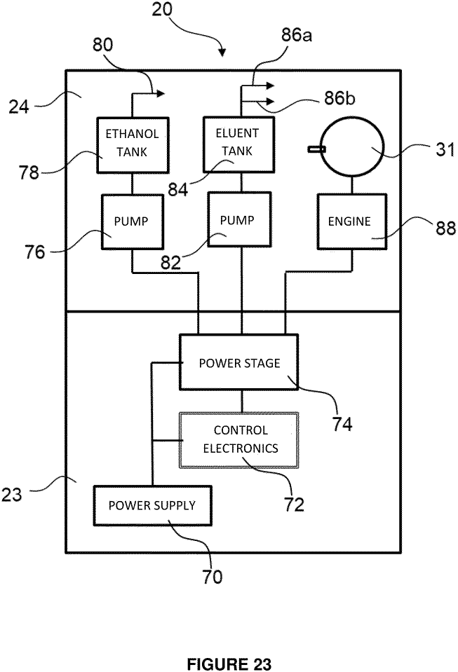

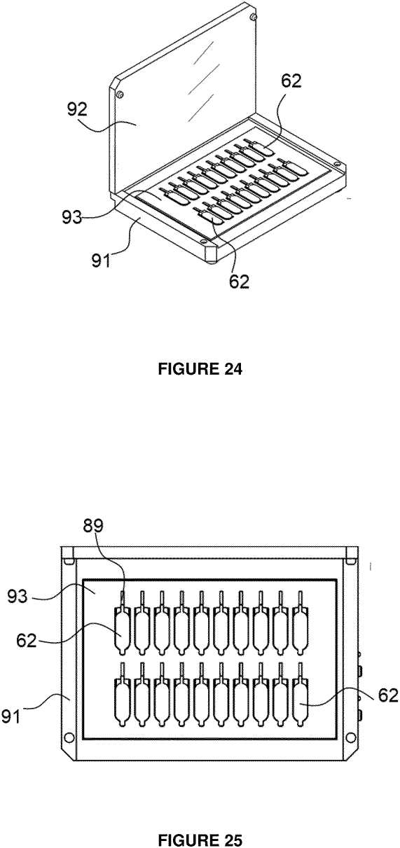

FIG. 23 shows schematically and by way of example a type of arrangement implemented in a dispenser (20) according to the invention.

Thus the base (23) comprises a power supply (70) which supplies energy to control electronics (72), controlled for example by a microcontroller, which controls, via a power stage (74), a first metering pump (76) in relation to a reservoir of ethanol (78) so as to inject the latter by means of a nozzle (80) into the cavity (50) of the diagnostic device having received the sample.

The base (23) also comprises a second metering pump (82) connected to an eluent reservoir (84) so as to inject the eluent by means of two nozzles (86a and 86b) onto the capture membranes (38) respectively contained in the openings (52a and 52b) of the diagnostic device (31).

The base (23) also includes a motor (88), for example a stepper motor, which is able, under the control of the control electronics, to position the upper disc in each of the three aforementioned positions as explained above.

Under these conditions the operation of the dispenser (20) is as described below. described below.

Once the user has taken the sample from the diagnostic device (31), he opens the cover (24) of the dispenser (20) and inserts the diagnostic device (31) with the open capsule (62) into the circular cavity of the base (23), then closes the cover (24) and starts the process by pressing an actuator (90).

The control electronics then manage the following phases:
1. the upper disc (33) of the diagnostic device (31) is in the aforementioned “first position” and the metering pump (76) then injects via its nozzle (80) into the cavity of the hollow boss (50) a quantity of the order of 400 µL of 70% ethanol so as to carry out a rinsing of this cavity;
2. the motor (88) then positions the upper disc (33) in the aforementioned “Intermediate position”, so as to bring the capture membranes (38a and 38b) in line with the radial window (58) so as to carry out a drying operation (axis r1 in line with axis r′2);
3. the control electronics (72) then apply a pause of the order of 3 to 15 minutes so as to ensure drying, which drying may be activated/promoted by blowing means, e.g., a pump blowing air through the capture membranes (38a and 38b);
4. the motor (88) then positions the upper disc (33) in the aforementioned “second position”, so as to bring the capture membranes (38a and 38b) in line with the reactive pastilles (43a and 43b) (axis r1 in line with axis r′3) and the nozzles (86a and 86b) inject a volume of between 30 µL and 50 µL of a saline solution onto each capture membrane (38a and 38b);
5. the motor (88) then returns the upper disc (33) to said “first position” or starting position; and
6. the operator has to open the cover (24), recover the capsule (62) and close it again with its cover (62b).

The present dispenser (20) is particularly interesting insofar as it allows such a treatment to be carried out automatically in a limited time of the order of 3 to 15 minutes. It can therefore of course be used on its own, but it can also be used in combination with a detector (21) allowing the detection of the nucleic acids contained in the biological sample collected, the combination of the dispenser (20) and the detector (21) making it possible to constitute two complementary devices forming a diagnostic system (22).

As shown in FIG. 24, the detector (21), which in this embodiment is in the form of a housing, has a body (91) and a cover (92) hinged thereto.

The interior of the body (91) comprises a plate (93) which comprises two rows of (separate and independent) cells for receiving the capsules (62). In the present example the plate comprises twenty cells but it is understood that it could comprise any number of cells.

The cover (92) is made of a transparent material, in particular a synthetic material which is dyed yellow.

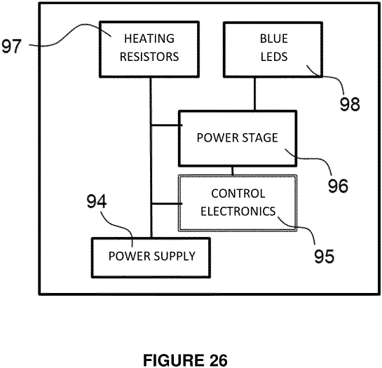

As shown in FIG. 26, the body (91) of the detector (21) comprises a power supply (94) which supplies energy to control electronics (95), controlled for example by a microcontroller, which controls, via a power stage (96), on the one hand, heating resistors (97) and, on the other hand, diodes (98) emitting blue radiation.

Under these conditions the operation of the detector (21) is as follows described below:
1. the user, after having opened the power supply (94) of the detector (21), inserts into one of the cells (89) of the latter a capsule (62) having undergone a treatment of the type previously carried out in the dispenser (20);
2. the control electronics (95) detects the cell which has just received the capsule (62) and applies to the latter, by means of a resistor (97), localised heating to a temperature of the order of 65° C. for a period of the order of 45 minutes;
3. at the end of this period, the electronic control system lowers the temperature of this cell to a value of 25° C. and controls, under this cell, the lighting of one or more blue LEDs (98) (light-emitting diodes) at the same time as it indicates the end of the process by the emission of a signal, in particular an acoustic signal;
4. through the yellow colored transparent cover the user can see the capsule (62) which is illuminated in blue from below, so that the fluorescence is clearly visible and the result can be easily read; and
5. the user can then open the power supply (94) and remove the capsule (62).

An average detection cycle is therefore between approximately 45 and 70 minutes.

The use of the detection system (22) according to the invention, i.e. the joint use of the dispenser (20) and the detector (21), is particularly interesting in that it allows them to be used sequentially in time-sharing mode.

As the average processing time in the dispenser (20) is of the order of 3 to 15 minutes and that in the detector of the order of 45 minutes, it is possible to process several samples in the dispenser during one processing cycle in the detector (21).

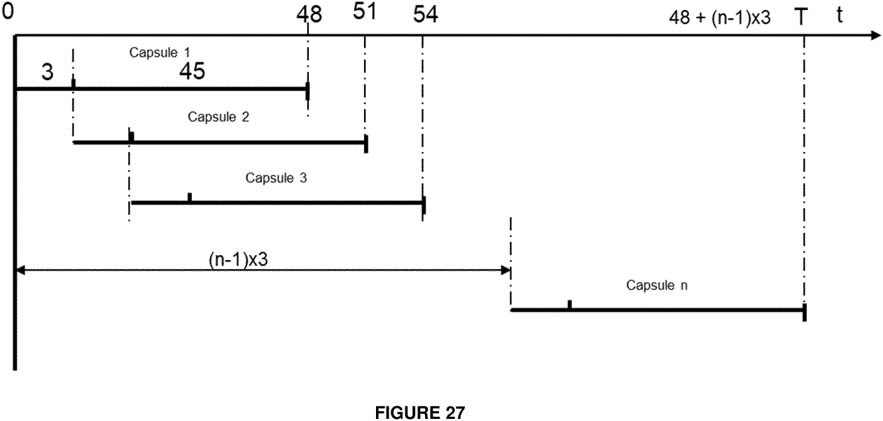

Thus, and considering as an example a processing time of 3 minutes for the dispenser (20) and a time of 45 minutes for the detector (21), the operating diagram shown in FIG. 27 is obtained.

It is thus found that the time to obtain the full results of the processing of n capsules (62), via the joint use of a dispenser and a detector capable of accommodating n capsules (62), is:

$$\text{T} \text{=48+} \left[\left(\text{n-1}\right) \times 3\right] .$$

This means that for 20 capsules in the detector: T = 105 minutes.

Finally and in view of the above, it is understood that another aspect of the invention relates to a portable diagnostic device (31) in the form of a cylindrical housing comprising two superimposed coaxial discs (33, 35) mounted for rotation with respect to each other about their centres (O) and defining a closed volume, namely:
an upper disc (33) comprising:
   at least one opening (52a, 52b) receiving a biological sample to be tested which is provided with a biologically compatible capture membrane (38) and capable of allowing the extraction and retention of nucleic acids from said biological sample in the presence of appropriate reagents, and
   at least one window (53), the receiving opening (52a, 52b) and this window (53) being angularly separated by an angle at the centre (α) of at least 30°,
a lower disc (35) comprising:
   an absorbent material (45) capable of containing a volume of liquid, and
   at least one capsule (62) removably disposed in a housing (56) thereof and which comprises a reaction zone comprising at least one and preferably at least two biologically compatible, adjacent, independent pastilles (43a, 43b) capable of permitting the amplification and detection of at least one nucleic acid sequence of interest likely to be present in the abovementioned biological sample in the presence of appropriate reagents,
the receiving opening (52a, 52b), the window (53), the absorbent material (45) and the reaction zone forming a detection unit such that, by rotation, the upper disc (33) can successively
from a first position in which, for this detection unit, its receiving opening (52a, 52b) is in line with its absorbent material (45), so as to allow the extraction and retention of said nucleic acids, and its window (53) is in line with its reaction zone,
at a second position in which, for this detection unit, its receiving opening (52a, 52b) is in line with its reaction zone and covers it in part or in full so as to allow elution of the abovementioned nucleic acids, and
position itself, by a rotation, on the aforementioned first position, so that, for this detection unit, its window (53) is in line with its reaction zone.

According to another embodiment, the invention concerns the diagnostic device (31) as described above, characterized in that the lower disc (35) comprises a drying window (58), in particular of radial shape, arranged between the housing (56) of the capsule (62) and the absorbent material (45), preferably at the same angular distance (α/2) from these two elements, this drying window (58) being arranged in such a way that, by a rotation, the upper disc (33) can pass from the above-mentioned first position to this intermediate position in which, for the above-mentioned detection unit, its receiving opening (52a, 52b) is located in line with this drying window (58).

According to another embodiment, the invention relates to the diagnostic device (31) as described above, characterized in that the housing suitable for receiving the capsule (62) is in the form of a radial slot (56) open to the outside, into which said capsule is slidably insertable.

According to another embodiment, the invention concerns the diagnostic device (31) as described above, characterized in that the capsule (62) comprises a first part (62a) capable of being inserted into the radial slot (56) and a second part forming a cover (62b) for closing off the first part, these two parts being possibly connected by connecting means (62c).

According to another embodiment, the invention relates to the diagnostic device (31) as described above characterized in that said first part (62a) and said second part (62b) is of transparent type, and in particular said first part (62a) or said second part (62b) is tinted with yellow color. It should be noted that since the detection of the signal obtained through the use of the diagnostic device of the invention is based on the principles of fluorescence, only one or the other of the parts (62a) and (62b) can be dyed yellow, which part is determined by the location of the light source.

For example, a light source (an LED) of a certain color (i.e. centred on a blue wavelength of 495 nm) illuminates a fluorophore (i.e. present on the pastilles and active at the end of the reaction in case of a positive result). The fluorophore in question, under the stimulation of the light source, then emits light of another color (i.e. of a green wavelength of the order of 517 nm) whose intensity is much weaker than the intensity emitted by the light source. It is therefore recommended or necessary to use a color filter (i.e. yellow) to block the light source but not the light emitted by the fluorophore. In this way, the signal can be observed/measured and the diagnosis made. As the color filter is to be placed between the observed biologically compatible side of the pastilles (43a, 43b) and the measuring device (e.g. eye or camera) to eliminate the light source and allow the fluorescence emitted by the fluorophore to pass through, which can then be detected, it is understood that this filter may be constituted by said first part (62a) or said second part (62b) of the transparent capsule, or by the cover (92) of the detector (21) if the latter is used. In other words:
if the holder (62a) of the capsule (62) is transparent and yellow, then the cover (62b) is transparent (without color) and the light source is above the capsule (62), facing the transparent cover (62b); and
if the cover (62b) of the capsule (62) is transparent and yellow, then the holder (62a) is transparent (without color) and the light source is below the capsule (62), facing the transparent holder (62a).

Note that this particular embodiment of the capsule (62) is in addition to the case where the detector (21) is not used, or if it is used, the cover (92) is not tinted yellow. Conversely, if the detector (21) as described above is used and includes a transparent yellow cover (92), then the entire capsule (62) is transparent and the light source is below the capsule. Beyond this example based on blue excitation and green emission, it is possible to adapt the color of the dyed materials to adapt the diagnostic device of the invention to the use of intercalating agents or fluorophores using different ‘excitation wavelength/emission wavelength’ pairs.

In particular and according to another embodiment, the invention relates to the diagnostic device (31) as described above characterized in that the capsule (62) is made of polypropylene, in particular food grade.

According to another embodiment, the invention relates to the diagnostic device (31) as described above, characterized in that the opening (52a, 52b) for receiving the biological sample to be tested is arranged at the bottom of a hollow boss (50), and comprises a removable funnel (49) provided with at least one opening located in line with the receiving opening (52a, 52b), this funnel (49) being able to be fitted onto the hollow boss (50).

According to another embodiment, the invention relates to the diagnostic device (31) as described above characterized in that the absorbent material (45) is either disposed in a small tank (59) of the upper surface of the lower disc (35) or integrated into the mass of the latter, said absorbent material (45) having a retention volume of at least 1 mL.

According to another embodiment, the invention relates to the diagnostic device (31) as described above characterized in that the absorbent material (45) is made of a material selected from: an absorbent material made of cellulose, glass fibre or cotton flock and an absorbent material suitable for use in an immuno-chromatographic test.

According to another embodiment, the invention relates to the diagnostic device (31) as described above characterized in that one of the two facing faces of one of the discs (33) comprises a groove (51) coaxial with the disc and partially circular, the ends of which determine an angle at the centre (β), the value of which, in cooperation with a tenon (60) of the other disc (35), define end position stops of the two discs in both said first and second positions.

According to another embodiment, the invention relates to the diagnostic device (31) as described above, characterized in that the biologically compatible capture membrane (38) capable of allowing the extraction and retention of nucleic acids is made of a material selected from: cellulose, silica and glass fibre.

According to another embodiment, the invention relates to the diagnostic device (31) as described above, characterized in that the at least one and preferably at least two biologically compatible amplification pastilles (43a, 43b) are made of a material selected from: glass fibre (whether or not containing a binder adjuvant) and cellulose.

According to another embodiment, the invention relates to the diagnostic device (31) as described above characterized in that at least one reaction zone comprises at least one preferably at least two biologically compatible, adjacent and independent amplification pastilles (43a, 43b), at least one of which contains lyophilized reagents capable of allowing the amplification and detection of at least one nucleic acid sequence of interest which may be present in the above-mentioned biological sample.

According to another embodiment, the invention relates to the diagnostic device (31) as described above characterized in that said at least one reaction zone comprises at least two biologically compatible, adjacent and independent amplification pastilles (43a, 43b),
the first (43a or 43b) containing lyophilized reagents capable of allowing the amplification and detection of at least one nucleic acid sequence of interest likely to be present in said biological sample and
the second (43b or 43a) containing lyophilized reagents capable of allowing the amplification and detection of at least one other nucleic acid sequence of interest necessarily present in the biological sample to be tested.

According to another aspect of the invention, the invention relates to the use of the portable diagnostic device (31) in the form of a cylindrical housing as described above to detect in vitro at least one nucleic acid sequence of interest likely to be present in a biological sample to be tested. According to another embodiment, the invention relates to the above-mentioned use for detecting in vitro a viral infection and in particular for detecting in vitro a SARS-CoV-2 viral infection.

According to another aspect of the invention, the latter concerns a method of in vitro detection of at least one nucleic acid sequence of interest likely to be present in a biological sample to be tested, said method being implemented with the aid of the portable diagnostic device (31) in the form of a cylindrical housing as described above and said method comprising at least the following steps:
a. extracting nucleic acids from the biological sample to be tested onto a biologically compatible capture membrane (38) of a detection unit;
b. rotating the upper disc (33) to position said biologically compatible capture membrane (38) over the reaction zone of said detection unit and partially or fully cover it;
c. eluting the nucleic acids from said biologically compatible capture membrane (38) to the at least one preferably at least two biologically compatible pastilles (43a, 43b) of said detection unit;
d. turning the upper disc (33) to return to the initial position.

According to another embodiment, the invention relates to the in vitro detection method as described above, wherein a step of drying said biologically compatible capture membrane (38) is performed between said steps a) and b), and said step comprises at least the following step:

i. turning the upper disc (33) to position said biologically compatible capture membrane (38) at the radial window (58) so as to achieve drying.

According to another aspect of the invention, the latter concerns a dispensing device (20) intended to implement in an automated manner a diagnostic device (31) as described above, characterized in that it comprises a receiver housing (23, 24) receiving:
a diagnostic device (31),
means for rotating (88) the upper disc (33) relative to the lower disc (35) of this diagnostic device (31),
means (72) for managing the relative positioning of its two discs (33, 35),
means for flushing (76, 78, 80) the at least one receiving opening (52a, 52b),
means for eluting (82, 84, 86a, 86b) the at least one capture membrane (38).

According to another embodiment, the invention concerns the dispensing device (20) as described above, characterized in that the management means comprise a power supply (70) delivering energy to a power stage (74) under the control of control electronics (72).

According to another embodiment, the invention concerns the dispensing device (20) as described above, characterized in that the control electronics (72) ensure the control of a motor (88), in particular a stepping motor, capable of sequentially positioning the upper disc (33) with respect to the lower disc (35) successively in the aforementioned first position, in the aforementioned intermediate position, in the aforementioned second position and in the aforementioned first position.

According to another embodiment, the invention relates to the dispensing device (20) as described above characterized in that it comprises means for drying (58) the capture membrane (38).

According to another embodiment, the invention relates to the dispensing device (20) as described above characterized in that the drying means comprise a radial slot (58) passing through the lower disc (35) and which is positioned so that it is in line with the at least one receiving opening (52a, 52b) when the upper (33) and lower (35) discs are in the above-mentioned intermediate position, and optionally blowing means through said radial slot (58).

According to another embodiment, the invention concerns the dispensing device (20) as described above, characterized in that the rinsing means comprise at least one reservoir (78) of liquid product, in particular ethanol and in particular 70% ethanol, making it possible to inject, by means of at least one pump (76), in particular a metering pump, and at least one nozzle (80), a quantity of the said product, in particular of the order of 400 µL, into the at least one receiving opening (52a, 52b). By “of the order of 400 µL” is meant a quantity of between 350 and 450 µL, in particular a quantity of between 375 and 425 µL, 390 to 410 µL or even a quantity of 400 µL.

According to another embodiment, the invention concerns the dispensing device (20) as described above, characterized in that the elution means comprise at least one reservoir (84) of eluting product, in particular a salt solution, making it possible to inject by means of at least one pump (82), in particular a metering pump, and at least one and preferably two nozzles (86a, 86b), a quantity of the said product, in particular of the order of 30 µL to 50 µL, into the at least one receiving opening (52a, 52b). By “of the order in particular of 30 µL to 50 µL” is meant a quantity of between 30 and 50 µL, in particular a quantity of between 30 and 40 µL, 40 to 50 µL or 35 to 45 µL; or even a quantity of 36 µL.

According to another aspect of the invention, the latter concerns a method of operating a dispensing device (20) as described above, characterized in that it comprises the steps in which the control electronics (72) successively ensure:
(the discs being in the above-mentioned first position), activating the at least one pump (76) in order to inject, through the at least one nozzle (80), ethanol contained in the at least one ethanol reservoir (78) into the at least one receiving opening (52a, 52b) in a quantity of the order of 400 µL in particular,
the relative positioning of the upper (33) and lower (35) discs in said second position,
injecting, in this second position, through at least one and preferably two nozzles (86a, 86b), the eluting product contained in the at least one reservoir (84) into the at least one receiving opening (52a, 52b) in a quantity of the order of 30 µL to 50 µL in particular,
the return of the upper (33) and lower (35) discs to the above first relative position.

According to another embodiment, the invention relates to the above method characterized in that the control electronics (72), when the upper disc (33) leaves the above first position for the above second position:
ensures the relative positioning of the upper (33) and lower (35) discs in said intermediate position,
controls a stop in this position, for a biologically compatible drying time of the capture membrane (38), in particular between a few seconds and fifteen minutes.

By “between a few seconds and fifteen minutes” is meant that the drying time can be between 1 and 60 seconds, 1 and 30 seconds, 1 and 15 seconds, 1 and 5 seconds, 1 and 15 minutes or 1 and 5 minutes. In particular, if drying means are used, a few seconds (1, 2, 3, 4, 5, 6, 7, 8, 9, 10 or 15 seconds) are sufficient to perform the drying step. By “drying means” is meant in particular blowing means.

According to another aspect of the invention, the invention relates to a test system characterized in that it comprises a diagnostic device (31) as described above and a dispensing device (20) as described above.

According to another aspect of the invention, it concerns a detector device (21) intended to ensure the detection of nucleic acids contained in a biological sample, for example collected in a diagnostic device (31) as described above and implemented in a dispensing device (20) as described above, this detector device (21) comprising a housing (91) comprising:
a plate (93) with a series of cells (89) for receiving capsules (62),
means for heating these capsules (62),
means for detecting a fluorescence of these capsules (62),
means for managing the treatment process (94, 95, 96).

According to another embodiment, the invention relates to the detector device (21) as described above, characterized in that the process management means comprise a power supply (94) providing energy to a power stage (96) under the control of control electronics (95).

According to another embodiment, the invention concerns the detector device (21) as described above, characterized in that the means for heating the capsules (62) consist of heating resistors (97) preferably disposed under each of the capsules (62) and capable of bringing the latter to a temperature in particular of the order of 65° C. By “of the order of 65° C.” is meant a temperature of 60 to 70° C., 62 to 68° C., 64 to 66° C. or a temperature of 65° C.

According to another embodiment, the invention relates to the detector device (21) as described above, characterized in that the fluorescence detection means comprise illumination means, in particular constituted by light-emitting diodes (LEDs) (98), which are arranged under the capsules (62) and by a yellow filter arranged above them.

According to another embodiment, the invention concerns the detector device (21) as described above, characterized in that the yellow filter is constituted by the cover (92) of the housing (91).

According to another aspect of the invention, the latter concerns a method for implementing a detector device (21) as described above, characterized in that it comprises the steps in which:
the user places one or more capsules in the cells (89) of the board (93) of the detector device (21),
the control electronics (95) activate the heating of the resistors (97) arranged under the capsule(s) (62) just inserted into the sensor device (21) to a value of approximately 65° C.,
the control electronics (95) maintains this temperature below the said capsule(s) for a period of approximately 45 minutes,
the control electronics (95) then lowers the temperature applied to said capsule(s) to a value of approximately 25° C.,
the control electronics then emit a signal, in particular an acoustic signal, indicating the end of the treatment for this or these capsule(s) and illuminate a blue light underneath it or them,
the user then places a yellow filter on the above-mentioned treated capsule(s) and in particular the transparent yellow-tinted cover (92), in order to detect any fluorescence,
the user then removes the treated capsule(s) from the sensor device (21).

According to another embodiment, the invention relates to the above method in which the fluorescence (the signal) is monitored in real time. For this purpose and among the reagents lyophilized on the biologically compatible pastilles (43a, 43b), probes coupled to a fluorophore are not used but probes without modification and an intercalating agent. It should be noted that with this real-time monitoring, the method of the invention allows the amplification kinetics to be followed and, from this, a semi-quantitative estimate of, for example, the viral load to be made; i.e. the user is able to estimate (relatively) in which of the samples being tested the viral load is the highest or lowest.

According to another aspect of the invention, the invention concerns a diagnostic system characterized in that it comprises a test system as described above and a detector device as described above.

In any event, it is recalled that the various aspects of the invention, as well as the various embodiments thereof, are interdependent. These can therefore be combined with each other in abundance to obtain preferred aspects and/or embodiments of the invention not explicitly described. This also applies to the set of definitions provided in this description, which applies to all aspects of the invention and its embodiments.

### EXAMPLES

The following examples illustrate the invention without limiting its scope.

### Example 1 - Manufacture of the Portable Diagnostic Device in the Form of a Cylindrical Housing of the Invention

### Creating Discs

Using the Fusion 360 software licensed by Autodesk, two circular pieces with a diameter of 65 mm and 58 mm were created, hereafter referred to as the upper and lower discs respectively.

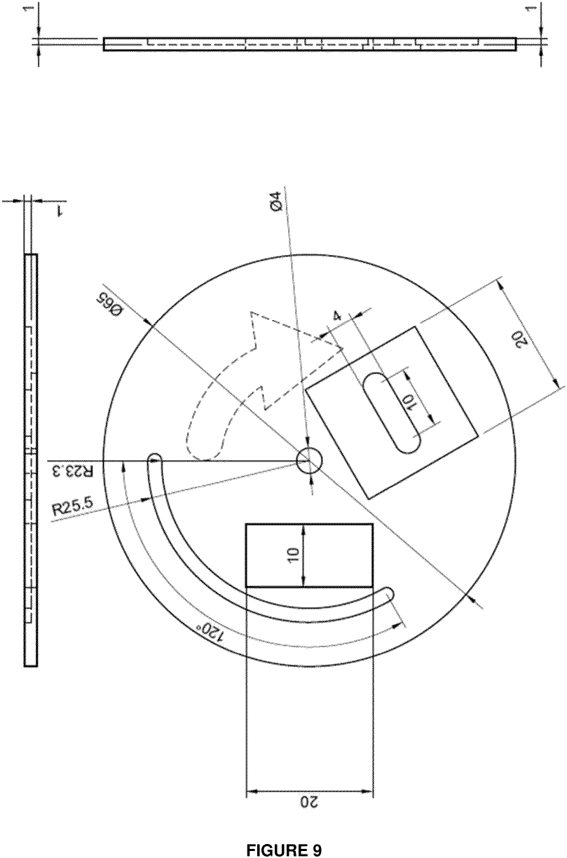

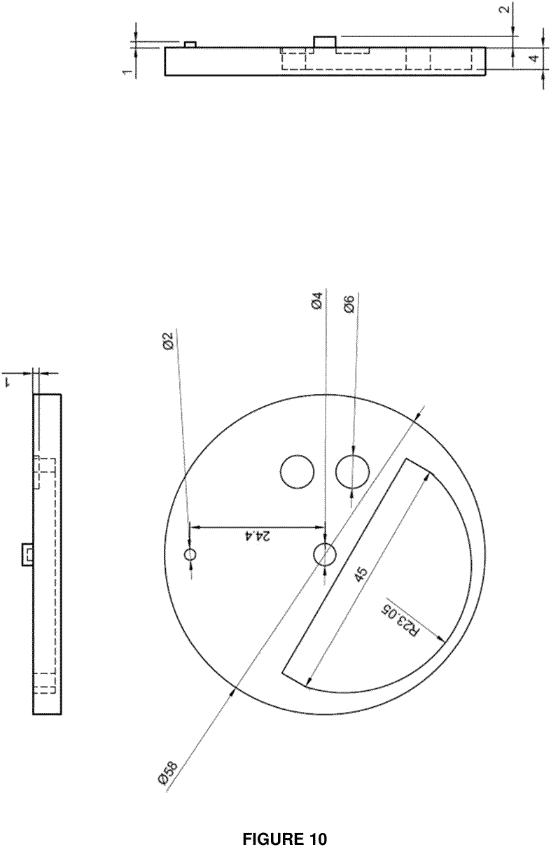

On the upper disc, the dimensions of drawing 1 in FIG. 9 have been reproduced. On the lower disc the dimensions of drawing 2 shown in FIG. 10 have been reproduced.

The dimensions of the drawings are in millimetres (mm).

The 3D parts of the 2 disks were exported in STL format and implemented in Makerboot software.

Printing with the Replicator 2X printer has been configured with the following print settings:
Nozzle temperature: 210° C.
Plateau temperature: 50° C.
Coating thickness: 100 µm
Writing speed: 110 mm/s
Filling: 45
Filling technique: Hexagonal

The print paths in 3gs format were exported and implemented in the printer and printing was started.

(5) After 3 hours of printing, the 2 discs were recovered by detaching them from the printing plate.

### Assembling the Different Elements on the Discs

After cleaning the upper disk with RNA free solution:
double-sided tape was applied to the contours of the two openings (3 mm wide), the taped side being the inside of the upper disc where the guide groove exists;
the biologically compatible nucleic acid extraction and retention membrane, the so-called capture membrane, has been arranged at the elongated hole-shaped receptor opening, said capture membrane having been previously encapsulated in PCR tape perforated on each side; and
The assembly was held together by pressing on the contours of the oblong hole to ensure the best possible contact between the tape and the upper disc.

Operation (1) was repeated with the optical filter previously cut and assembled with a heat-shrinkable PCR sheet for the side in contact with the upper disc.

After cleaning the lower disc with RNA free solution:
the absorbent material, known as a capillary pad, has been placed in the semicircular receptacle (or cavity); and
The 2 amplification and detection pastilles have been placed in the circular recesses provided for this purpose in the reaction zone, which recess in particular allows the light to reach the said pastilles.

### Disc Assembly

The upper and lower discs have been assembled, taking care to ensure that the stop pin is in the guide groove and that the hole in the centre of the upper disc is aligned with the centre stud of the lower disc.

The ready device can either be stored and used later or used directly.

### In Situ Lyophilization of Reagents

In addition to the manufacture of the diagnostic device of the invention, a step of freeze-drying the molecular tools allowing the amplification and detection of at least one nucleic acid sequence likely to be present in the sample to be tested can be added. For this purpose, dNTPs, enzymes, primers, etc. are mixed and adsorbed onto the pastilles. The reaction pastilles are then incubated at -20° C. for 20 minutes, then at -80° C. for 20 minutes, and then freeze-dried in a freeze-dryer at a pressure of 3 mbar overnight (8 h). The pastilles thus freeze-dried can then be placed in the housings provided for this purpose in the device of the invention.

### Example 2 - Uses of the Diagnostic Device of the Invention for Diagnosing Dengue Virus Infection and SARS-COV-2 Coronavirus Infection

### Materials & Methods

### Lyophilisation of Compounds

The following compounds were lyophilized in each tablet (18 µL):
0.85 mM of each deoxyribonucleotide triphosphate (dATP, dTTP, dGTP, dCTP)
5 mM MgSO₄
0.5 mM Betaine
0.2 µM of each primer F3 and B3
0.8 µM of each FIP and BIP primer
0.4 µM of each LoopF and LoopB primer
a quencher concentration one and a half times higher than the concentration of the probe primer (1.2 µM if the probe primer is FIP or BIP, 0.6 µM if the probe primer is LoopF or LoopB)
7.2 units of GspSSD2.0 polymerase enzyme
0.9 units of AMV-RT retro-transcriptase enzyme
5% v/v Trehalose, allowing the stability of the lyophilisate.

For the detection of DENV2, primers with sequence SEQ ID NOs: 1-6 were used, which amplify a target sequence of the NS4B gene, and the BIP primer (SEQ ID NO: 4) was used as a probe as it was modified in the 5′ by the addition of a FAM fluorophore. The sequence oligonucleotide-quencher SEQ ID NO: 13 was used and modified in the 3′ by the addition of Iowa Black FQ.

For the detection of the SARS-CoV-2 coronavirus, primers with sequences SEQ ID NOs: 7 to 12 were used, which amplify a target sequence of the ORF1ab gene (Lin Yu et al. Rapid colorimetric detection of COVID-19 coronavirus using a reverse transcriptional loop-mediated isothermal amplification (RT-LAMP) diagnostic plat-form: iLACO; DOI-10.1101/2020.02.20.20025874), and the LoopF primer (SEQ ID NO: 11) was used as a probe as it was modified in the 5′ direction by addition of a Texas Red fluorophore. The sequence oligonucleotide-quencher SEQ ID NO: 16 was used and modified in the 3′ by the addition of Iowa Black RQ.

These primers (etc.) were lyophilized (see above) in a test pastille alongside an internal positive control pastille in which the template DNA/RNA to initiate the reaction was also lyophilized.

After freeze-drying, the pastilles were placed in the appropriate compartments.

### Use of the Device

Extraction of nucleic acids from the sample
Mix 100 µL of sample with AVL buffer (Qiagen®) or any other lysis buffer with a composition close to the published lysis buffer: Boom et al., J. P. M. E. (1990). Rapid and simple method for purification of nucleic acids. Journal of clinical microbiology, 28(3), 495-503
Vortex for 15 seconds
Incubate for 5 minutes at 65° C.
Add 400 µL of pure ethanol
Vortex for 15 seconds
Deposit the lysate in the capture zone, possibly using a funnel
(Remove the funnel)
Rinse the capture membrane with one or more rinse buffers: e.g. 200 µL of Buffer AW1 (Qiagen®), followed by 200 µL of Buffer AW2 (Qiagen®), or 400 µL of 70% ethanol solution, or any other rinse buffer with a composition close to that of the published rinse buffer: Boom et al. Rapid and simple method for purification of nucleic acids. Journal of clinical microbiology, 28(3), 495-503
Allow the capture membrane to dry for 15 minutes at 65° C.

Elution in reaction pastilles and heating step
Rotate the upper disc to face the capture membrane and pastilles, and rehydrate the capture membrane with 43 µL of 1X Buffer Reaction Buffer (Qiagen®) to allow elution of the nucleic acids
Turn the upper disc either in the same direction or in the opposite direction so as to cover the discs with the optical filter, in this case, a PCR tape
Incubate the device at 65° C. for 30 to 45 minutes, e.g. in an oven or on heaters.

For the purposes of this example, the samples used were a DENV2 infected sample and a DENV2 uninfected sample. A SARS-CoV-2 RNA extract and a negative control without RNA extract were also used.

### Data Acquisition

End-point fluorescence acquisition via the probes used and the associated quenchers was performed:
for DENV2 (fluorophore = FAM) with an excitation length of 495 nm and an emission wavelength of 520 nm; and
for the coronavirus SARS-CoV-2 (fluorophore = Texas Red) with an excitation wavelength of 586 nm and an emission wavelength of 603 nm.

Photographs were also taken with a smartphone (Iphone® ) or a commercial Nikon camera®.

### Results

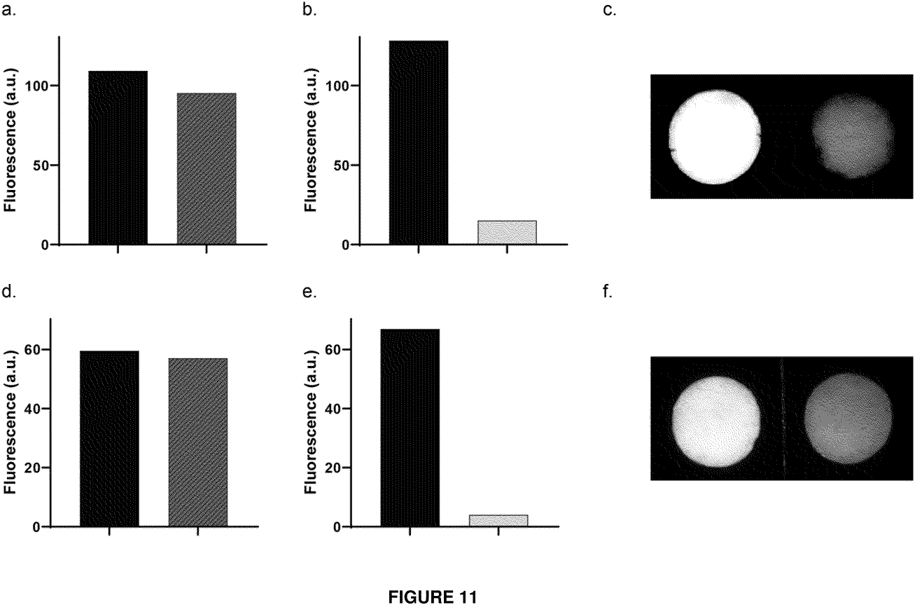

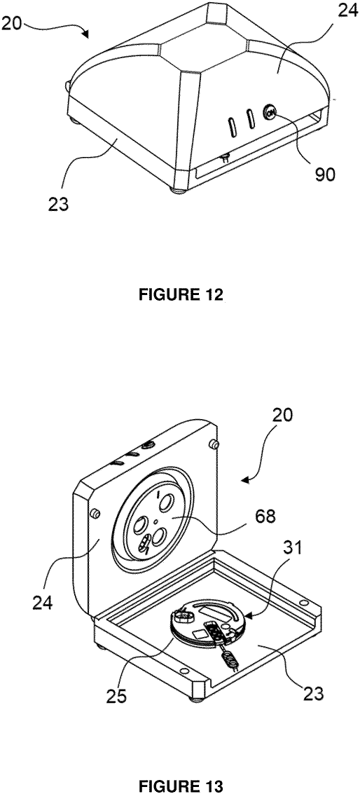

FIG. 11 shows the results obtained, which confirmed that the device is able to safely and reliably detect DENV2 and SARS-CoV-2 coronavirus only in samples where their respective genetic material is present.

### Example 3 - Uses of the Diagnostic Device of the Invention for SARS-COV-2 RNA in Artificial Saliva and Nasopharyngeal Fluid Samples

### Materials & Methods

The samples were provided by XPrize as part of the XPrize rapid covid testing competition and were tested blind before the concentration values were released by XPrize.

The device of the invention has been used and has been configured to allow detection of SARS-CoV-2 RNA (Orf1ab gene) [Lamb, L. E., Bartolone, S. N., Ward, E., & Chancellor, M. B. (2020). Rapid detection of novel coronavirus/Severe Acute Respiratory Syndrome Coronavirus 2 (SARS-CoV-2) by reverse transcription-loop-mediated isothermal amplification. PLoS One, 15(6), e0234682] as well as human 18S RNA [Lamb, L. E., Bartolone, S. N., Tree, M. O., Conway, M. J., Rossignol, J., Smith, C. P., & Chancellor, M. B. (2018). Rapid detection of Zika virus in urine samples and infected mosquitos by reverse transcription-loop-mediated isothermal amplification. Scientific reports, 8(1), 1-9]. The detection of 18S RNA allowed control of the sampling, extraction and storage of the device of the invention. On the upper disc, two circular silica membranes placed in the capture zone allow the capture of nucleic acids. On the lower disc, an absorbent material allows the flow to be directed by capillary action. Also on the lower disc is a resealable capsule containing 2 glass fibre detection membranes. One, round, contains a freeze-dried RT-LAMP mix for the detection of SARS-CoV-2 (SEQ ID NOs: 18 to 24). The other, square, contains a lyophilized RT-LAMP mix for the detection of 18S RNA (SEQ ID NOs: 25 to 31) [Garneret, P., Coz, E., Martin, E., Manuguerra, J. C., Brient-Litzler, E., Enouf, V., ... & Tabeling, P. (2021). Performing point-of-care molecular testing for SARS-CoV-2 with RNA extraction and isothermal amplification. Plos one, 16(1), e0243712. 7].

TABLE 2

Sequences of primers used for the detection of SARS-CoV-2 and human 18S ribosomal RNA (positive extraction control)

| PRIMERS | 5′ TO 3′ SEQUENCE | SEQ ID NO: |
|---|---|---|
| Covid-19_F3 | TCCAGATGAGGATGAAGAAGA | 18 |
| Covid-19_B3 | AGTCTGAACAACTGGTGTAAG | 19 |
| Covid-19_FIP | AGAGCAGCAGAAGTGGCACAGGTGATTGTGAAGAGAAGAGAG | 20 |
| Covid-19_BIP | TCAACCTGAAGAAGAGCAAGAACTGATTGTCCTCACTGCC | 21 |
| Covid-19_LoopF | CTCATATTGAGTTGATGGCTCA | 22 |
| Covid-19_LoopB | ACAAACTGTTGGTCAACAAGAC | 23 |
| Covid-19_FIP-Deactivator | GCTGCTCT | 24 |
| 18S_rRNA_F3 | GTTCAAAGCAGGCCCGAG | 25 |
| 18S_rRNA_B3 | CCTCCGACTTTCGTTCTTGA | 26 |
| 18S_rRNA_FIP | TGGCCTCAGTTCCGAAAACCAACCTGGATACCGCAGCTAGG | 27 |
| 18S_rRNA_BIP | GGCATTCGTATTGCGCCGCTGGCAAATGCTTTCGCTCTG | 28 |
| 18S_rRNA_LoopF | AGAACCGCGGTCCTATTCCATTATT | 29 |
| 18S_rRNA_LoopB | ATTCCTTGGACCGGCGCAAG | 30 |
| 18S_rRNA_FIP-Deactivator | CGAATGCC | 31 |

The COVID-19_FIP primer is modified in the 5′ by the addition of a FAM molecule, and a complementary oligo with a deactivator in the 3′ is added (Covid-19_FIP-Deactivator). Similarly, the 18S_rRNA_BIP primer is modified in the 5′ direction by the addition of a FAM molecule, and a complementary oligo with a deactivator in the 3′ direction is added (18S_rRNA_BIP-Deactivator).

The sample (100 µL) is lysed for 5 min at 65° C. The two discs forming the device are arranged so that the capture membranes are placed on top of the absorbent material. After adding 400 µL of 100% ethanol, this volume is deposited in the capture zone. The funnel is removed and 400 µL of a 70% ethanol solution is deposited in the capture zone to rinse the capture membranes. The upper disc is rotated so that the capture membranes on the upper disc face the drying zone of the lower disc. The capture membranes then dry for 15 minutes at 65° C. The upper disc is rotated again to bring the two capture membranes opposite the two amplification membranes respectively. 40 µL of an aqueous solution is deposited on each of the two capture membranes, eluting the RNA captured by the amplification membranes. This eluate rehydrates the freeze-dried RT-LAMP mix on the detection membranes. The capsule is then sealed and heated to 65° C. for 45 minutes. The amplification product is then observed at room temperature by fluorescence.

Ct values are obtained by the IP4 reference rt-PCR [World Health Organization. Protocol: real-time RT-PCR assays for the detection of SARS-CoV-2, Institut Pasteur, Paris. World Health Organization, Geneva].

### Results

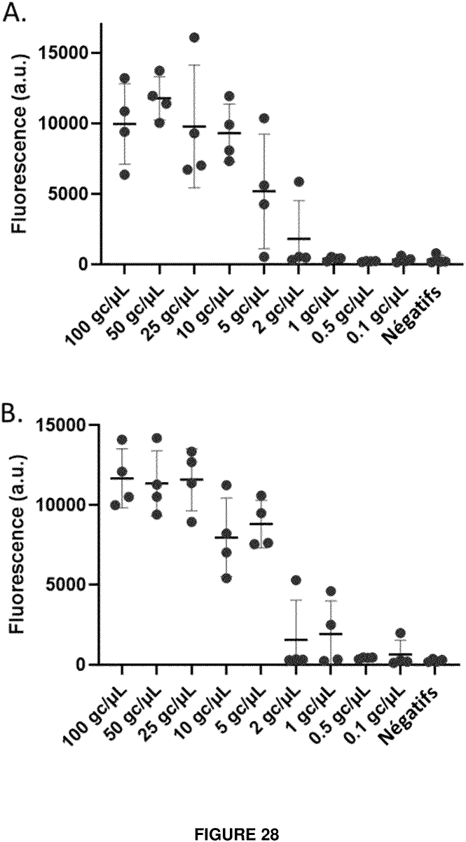

FIG. 28 shows the results obtained, which confirmed that the device is capable of safely and reliably detecting the SARS-CoV-2 coronavirus in nasopharyngeal and salivary samples.

In addition, the device has been shown to have excellent clinical sensitivity (i.e. 97%) and 100% specificity as illustrated in Table 3 below.

TABLE 3

Retrospective clinical trial for the characterization of the clinical sensitivities and specificities of the COVIDSIC rotary system

| Categories CI IP4 | COVIDISC (%,95% CI) | Total |
|---|---|---|
| Total positives | 36 (97.3, 85.8-99.9) | 37 |
| low (>30) | 14(93.3, 68.1-99.8) | 15 |
| medium (20-30) | 16 (100, 79.4-100) | 16 |
| high (<20) | 6 (100, 54.1-100) | 6 |
| Negatives | 62 (100, 94.2-100) | 62 |

Positive and negative concordance with IP4 reference PCR [World Health Organization. Protocol: real-time RT-PCR assays for the detection of SARS-CoV-2, Institut Pasteur, Paris. World Health Organization, Geneva]. 99 nasopharyngeal samples were tested, 37 positive for SARS-CoV 2 and 62 negative. The clinical sensitivity of COVIDISC was 97% (36/37) and the specificity 100% (62/62).

1-50. (canceled)

51. A portable diagnostic device in the form of a cylindrical housing comprising two superimposed coaxial discs mounted for rotation with respect to each other and defining a closed volume, namely:
an upper disc comprising:
at least one receiving opening of a biological sample to be tested which is provided with a biologically compatible membrane and capable of allowing the extraction and retention of nucleic acids from this biological sample in the presence of appropriate reagents, and
at least one window which is either equipped with an optical filter integrated in the mass of the upper disc or is able to receive an optical filter which can be positioned on said window in a removable manner,
said at least one receiving opening and said window being angularly spaced at an angle of at least 30° on centre,
a lower disc comprising:
an absorbent material capable of containing a volume of liquid, and
at least one reaction zone comprising at least one biologically compatible pastille capable of allowing the amplification and detection of at least one nucleic acid sequence of interest likely to be present in the abovementioned biological sample in the presence of appropriate reagents,
the receiving opening, the window, the absorbent material and the reaction zone form a detection unit such that, by rotation, the upper disc can successively
from a first position in which, for this detection unit, its receiving opening is in line with its absorbent material, so as to allow the extraction and retention of said nucleic acids, and its window is in line with its reaction zone,
at a second position in which, for this detection unit, its receiving opening is in line with its reaction zone and covers it in part or in full so as to allow the elution of the abovementioned nucleic acids, and
to position itself, by a rotation, on the above-mentioned first position, so that, for this detection unit, its window is in line with its reaction zone, so as to allow the amplification and the detection of the above-mentioned at least one nucleic acid sequence of interest likely to be present in the biological sample.

52. The diagnostic device according to claim 51, wherein the lower disc comprises at least one reaction zone comprising at least two independent, adjacent, biologically compatible pastilles capable of allowing the amplification and detection of at least one nucleic acid sequence of interest likely to be present in the abovementioned biological sample in the presence of appropriate reagents.

53. The diagnostic device according to claim 51, wherein the passage from said second position to said first position is made by a rotation opposite to that allowing the passage from said first position to said second position.

54. The diagnostic device according to claim 51, wherein the absorbent material is disposed in a receptacle which is positioned either on the upper surface of the lower disc or integrated into the mass of the latter.

55. The diagnostic device according to claim 53, wherein said absorbent material is able to hold a volume of at least 1 mL.

56. The diagnostic device according to claim 51, wherein the absorbent material is of a material selected from: a cellulose absorbent material, glass fibre or cotton flock and an absorbent material suitable for immuno-chromatographic testing.

57. The diagnostic device according to claim 51, wherein the biologically compatible membrane capable of allowing the extraction and retention of nucleic acids is of a material selected from: cellulose, silica and glass fibre.

58. The diagnostic device according to claim 51, wherein the at least one biologically compatible amplification pastille is of a material selected from: glass fibre (whether or not containing an adjuvant binder) and cellulose.

59. The diagnostic device according to claim 52, wherein the at least two biologically compatible amplification pastilles are of a material selected from: glass fibre (whether or not containing an adjuvant binder) and cellulose.

60. The diagnostic device according to claim 51, wherein at least one reaction zone comprises at least one biologically compatible amplification pastille,
at least one containing lyophilized reagents capable of allowing the amplification and detection of at least one nucleic acid sequence of interest likely to be present in the above-mentioned biological sample.

61. The diagnostic device according to claim 51, wherein at least one reaction zone comprises at least two biologically compatible, adjacent and independent amplification pastilles,
at least one containing lyophilized reagents capable of allowing the amplification and detection of at least one nucleic acid sequence of interest likely to be present in the above-mentioned biological sample.

62. The diagnostic device according to claim 51, wherein said at least one reaction zone comprises at least two adjacent and independent biologically compatible amplification pastilles,
the first containing lyophilized reagents capable of allowing the amplification and detection of at least one nucleic acid sequence of interest likely to be present in said biological sample and
the second containing lyophilized reagents capable of allowing the amplification and detection of at least one other nucleic acid sequence of interest necessarily present in the biological sample to be tested.

63. The diagnostic device according to claim 51, the diameter to height ratio of the device being from 5 to 30.

64. The diagnostic device according to claim 51, wherein said rotations are guided by a system of groove(s) and pin(s).

65. The diagnostic device according to claim 51, comprising:
at least two detection units which are distributed over the upper and lower discs in such a way that their respective receiving openings lie on the same radius of the upper disc, or
at least two detection units which are distributed over the upper and lower discs in such a way that their respective receiving openings are located on the same arc of the upper disc.

66. The diagnostic device according to claim 51, further comprising
at least one clamp capable of gripping said device and applying pressure to lock and clamp the upper disc to the lower disc, said pressure ensuring hermeticity and sealing of the device, and
equipped with a heating system capable of being positioned in line with the reaction zone(s) of the device avoiding the creation of a temperature gradient on the reaction zone(s) to be heated.

67. A method for the in vitro detection of at least one nucleic acid sequence of interest likely to be present in a biological sample to be tested, said method being implemented with the aid of the portable diagnostic device in the form of a cylindrical housing according to claim 51 and said method comprising at least the following steps:
(a) extracting nucleic acids from the biological sample to be tested onto a biologically compatible membrane of a detection unit;
(b) turning the upper disc to position said biologically compatible membrane over the reaction zone of that detection unit and cover it in part or in whole;
(c) eluting the nucleic acids from said biologically compatible membrane to the at least one biologically compatible pastille of said detection unit;
(d) turning the upper disc to return to the original position; and
(e) amplifying and then detect whether amplification of at least one nucleic acid sequence of interest that may be present in a biological sample has occurred.

68. A method of manufacturing the portable diagnostic device in the form of a cylindrical housing according to claim 51, said method comprising at least the following steps:
(a) manufacturing the upper and lower discs;
(b) disposing at least one biologically compatible membrane, at least one biologically compatible pastille, at least one absorbent material and at least one optical filter; and
(c) assembling the upper and lower discs.
